SEQUENCE LISTING (32 sequences)

SEQ ID NO: 1 - DNA, 40 bp, Artificial Sequence
  Artificially synthesized
gatgtggagtgcctgatgtggatgtggagtgcctgatgtg

SEQ ID NO: 2 - DNA, 20 bp, Artificial Sequence
  Artificially synthesized
tgcttgaccctcagagacct

SEQ ID NO: 3 - DNA, 20 bp, Artificial Sequence
  Artificially synthesized
tgaaggtcggtgtgaacgga

SEQ ID NO: 4 - DNA, 23 bp, Artificial Sequence
  Artificially synthesized
catgtagccatgaggtccaccac

SEQ ID NO: 5 - DNA, 20 bp, Artificial Sequence
  Artificially synthesized
tctgcaaggttatcccaagg

SEQ ID NO: 6 - DNA, 20 bp, Artificial Sequence
  Artificially synthesized
tattcctggaaaggcacctg

SEQ ID NO: 7 - DNA, 20 bp, Artificial Sequence
  Artificially synthesized
cactgtccacccctcagagc

SEQ ID NO: 8 - DNA, 18 bp, Artificial Sequence
  Artificially synthesized
cacttgtcggcgataagg

SEQ ID NO: 9 - DNA, 20 bp, Artificial Sequence
  Artificially synthesized
aattccgacctcgtcatcag

SEQ ID NO: 10 - DNA, 20 bp, Artificial Sequence
  Artificially synthesized
tgcagttttccagcaatgag

SEQ ID NO: 11 - DNA, 20 bp, Artificial Sequence
  Artificially synthesized
tgatccacacacgttggtct

SEQ ID NO: 12 - DNA, 20 bp, Artificial Sequence
  Artificially synthesized
tttgagttgcttgcaggatg

SEQ ID NO: 13 - DNA, 20 bp, Artificial Sequence
  Artificially synthesized
gagtcaacggatttggtcgt

SEQ ID NO: 14 - DNA, 20 bp, Artificial Sequence
  Artificially synthesized
gacaagcttcccgttctcag

SEQ ID NO: 15 - DNA, 20 bp, Artificial Sequence
  Artificially synthesized
ggagccagctccctctattt

SEQ ID NO: 16 - DNA, 20 bp, Artificial Sequence
  Artificially synthesized
ggctacatgggaacagccta

SEQ ID NO: 17 - DNA, 20 bp, Artificial Sequence
  Artificially synthesized
ctgtcctgcgtgttgaaaga

SEQ ID NO: 18 - DNA, 20 bp, Artificial Sequence
  Artificially synthesized
ttctgcttgagaggtgctga

SEQ ID NO: 19 - DNA, 20 bp, Artificial Sequence
  Artificially synthesized
aggagacttgcctggtgaaa

SEQ ID NO: 20 - DNA, 20 bp, Artificial Sequence
  Artificially synthesized
caggggtggttattgcatct

SEQ ID NO: 21 - DNA, 21 bp, Artificial Sequence
  Artificially synthesized
ggccattcctggtggagttcc

SEQ ID NO: 22 - DNA, 25 bp, Artificial Sequence
  Artificially synthesized
aactggcttaagaggtttgcctcca

SEQ ID NO: 23 - DNA, 20 bp, Artificial Sequence
  Artificially synthesized
cagtcgcttcacctacagca

SEQ ID NO: 24 - DNA, 20 bp, Artificial Sequence
  Artificially synthesized
ggtggagggagtttacacga

SEQ ID NO: 25 - DNA, 22 bp, Artificial Sequence
  Artificially synthesized
cgagtgccaaatgaagaggagc

SEQ ID NO: 26 - DNA, 22 bp, Artificial Sequence
  Artificially synthesized
aaacctgagccagaacctgacg

SEQ ID NO: 27 - DNA, 25 bp, Artificial Sequence
  Artificially synthesized
accatcgccttcgccctgctcatcc

SEQ ID NO: 28 - DNA, 25 bp, Artificial Sequence
  Artificially synthesized
cccgctccacattgaaggctaccca

SEQ ID NO: 29 - DNA, 30 bp, Artificial Sequence
  Artificially synthesized
tttctttatgtgactcatctgtctcaccgg

SEQ ID NO: 30 - DNA, 28 bp, Artificial Sequence
  Artificially synthesized
attgttggctaccagtttatccaaaggg

SEQ ID NO: 31 - DNA, 26 bp, Artificial Sequence
  Artificially synthesized
cagaaggctggacatatagaggaggg

SEQ ID NO: 32 - DNA, 27 bp, Artificial Sequence
  Artificially synthesized
attgttggctaccagtttatccaaacg

(19) United States
(12) Patent Application Publication
(10) Pub. No.: US 2013/0338221 A1
(43) Pub. Date: Dec. 19, 2013

(54) PHOTOAGING PROTECTIVE COMPOSITION CONTAINING CHROMENES DERIVED FROM SARGASSUM HORNERI

(71) Applicant: PUKYONG NATIONAL UNIVERSITY INDUSTRY-UNIVERSITY C, Busan (KR)

(72) Inventors: Se-Kwon KIM, Busan (KR); Jung-Ae KIM, Busan (KR)

(73) Assignee: Pukyong National University Industry-University Cooperation Foundation, Busan (KR)

(21) Appl. No.: 13/665,368

(22) Filed: Oct. 31, 2012

(30) Foreign Application Priority Data
Jun. 19, 2012 (KR) 10-2012-0065631

(51) Int. Cl. A61K 8/49; A61Q 19/08

(52) U.S. Cl. CPC A61K 8/498; A61Q 19/08
USPC 514/458; 549/407

(57) ABSTRACT

There is provided a photoaging protective composition having an effect of protecting photoaging on skin cell, the composition including chromenes derived from Sargassum horneri. Specifically, for the photoaging protective composition, sargachromanol D and sargachromanol E isolated from Sargassum horneri, brown algae exhibits activity of inhibiting MMPs, increase an expression of a collagen synthetic marker, such as procollagen and Type I collagen in fibroblasts treated with UV-A, or increases elastin by controlling elastase. For this reason, the chromenes derived from Sargassum horneri according to the present invention can be used as uses of a material or cosmetic products for improving and preventing skin wrinkles, the material having an effect of improving skin wrinkles caused by UV.

### CROSS-REFERENCE TO RELATED APPLICATIONS

This application claims the priority of Korean Patent Application No. 2012-65631 filed on Jun. 19, 2012, in the Korean Intellectual Property Office, the disclosure of which is incorporated herein by reference.

### BACKGROUND OF THE INVENTION

1. Field of the Invention

The present invention relates to a photoaging protective composition including chromenes derived from Sargassum horneri and cosmetic products for improving and preventing skin wrinkles including the same.

2. Description of the Related Art

As all living things get older, their skins are subjected to the aging process. The aging of skin can be largely divided into two groups according to their causes. As one of two groups, an intrinsic aging occurs by continuously decreasing a skin structure and physiological functions of skin as getting older. As the other group, an extrinsic aging occurs due to an accumulated external stress, like the sun's ray. Particularly, ultraviolet ray of sun light is one of aging causes that is well known. With respect to the skin exposed to UV rays for a long period time, since stratum corneum becomes thicker and collagen and elastin that are a main component of skin are denatured, skin's elasticity becomes get damaged and the skin becomes have wrinkles.

As disclosed above, the aging of skin involves various functional and structural changes. First, as for the structural change of skin according to aging, the thicknesses of epidermis, corium, and subcutaneous tissue that are constituents of skin become thinner. In addition, an extracellular matrix (ECM) component of corium tissue that has a key part in skin elasticity and tensile is changed. An extracellular matrix is largely composed of two components. One is a elastic fiber that occupies approximately 2% to 4% of the whole ECM and the other is collagen that occupies approximately 70% to 80% of the whole ECM. As the aging progresses, skin's elasticity significantly decreases because the collagen and elastin decrease. Such collagen and elastin can be controlled by various factors. Collagen and elastin generated by expressing matrix metallo protease, such as elastase, and collagenase, and as a result, there is a phenomenon in that the content of the collagen in skin is decreased. When the contents of collagen and elastin in corium are decreased, the epidermis of skin roughens, elasticity occurs as the aging process, and thus the skin has more wrinkles. Many researches on suppressing effectively the decreases of collagen and elastin that are a cause of decrease of elasticity are proceeding.

A method that is known as most effective method is to use retinol and retinoic acid. The effect of retinoid such as retinol and retinoic acid on improving wrinkles or elasticity is well known in document (Dermatology therapy, 1998, 16, 357˜364). However, such retinoid has a positive effect on improving wrinkles or elasticity, but also has a disadvantage in that even though only small amount of retinoid is applied on skin, irritation occurs. In addition, since the retinoid is unstable, when it is exposed to the air, it is easily oxidized and denatured. Accordingly, there are restrictions on the use of the retinoid. Thus, a research on stabilizing retinoid is continuously proceeding. However, a stability problem, i.e., a skin irritation is yet to be resolved until now. As a plan that can substitute for the above described method, the activities of enzymes capable of decomposing collagen and elastin are suppressed. Thus, a research about inhibitors capable of suppressing such enzymes is actively proceeding. Recently, with confirming that remarkably increased activities of collagenase and matrix metallo protease that are capable of decomposing collagen are one of skin wrinkles-generating factors, interest in such a result as a target for improving skin wrinkles is rising.

### SUMMARY OF THE INVENTION

An aspect of the present invention provides a new effective material for preventing photoaging, in which the new effective material exhibits an effect on improving wrinkles caused by UV rays and at the same time, has safety to human skin. Accordingly, the present inventors found that chromenes derived from Sargassum horneri have effect on stimulating a synthetic of collagen, suppressing a decomposition of collagen, and improving wrinkles induced by ultraviolet, and at the same time, have excellent safety to human skin. Accordingly, the present invention is completed.

Accordingly, an aspect of the present invention provides cosmetic products for improving and preventing skin wrinkles, in which the cosmetic products include chromenes derived from Sargassum horneri as an effective component, so that excellent effect on improving wrinkles can be obtained without any side effects.

Also, the present invention provides a method for protecting the skin photoaging in the skin of a mammal exposed to ultraviolet comprising administering to the mammal in need thereof chromenens selected from the group consisting of sargachromenol E, sargachromenol D, and a mixture thereof derived from Sargassum horneri as an effective component.

Also, the present invention provides a composition for inhibiting photoaging of human skin comprising chromenens selected from the group consisting of sargachromenol E, sargachromenol D, and a mixture thereof derived from Sargassum horneri as an effective component.

The present inventors measured and investigated a ROS production, a cytokine secretion, and an expressed aging-related gene expression in human dermal fibroblasts (HDF) irradiated with UV-A in order to confirm an effect of chromenes isolated from Sargassum horneri on a photoaging protection. The many conventional researches disclose that an activity of matrix metalloproteinases (MMPs) involve in a photoaging process. However, there were no researches on demonstrating a direct effect on a UV-mediated change in skin dermis in vitro. As a result, it was confirmed that a photoaging such as skin wrinkles caused by UV-A could be suppressed by treating chromenes derived from Sargassum horneri according to the present invention.

According to an aspect of the present invention, it was confirmed that the chromenes stimulate a synthesis of collagen and suppress a decomposition of collagen. Specifically, it is confirmed that the three chromenes suppress a decomposition of collagen, in which the decomposition was caused by expressing MMPs, MMP-1, MMP-2, MMP-3, and MMP-9. In addition, it is confirmed that such suppression occurs through increasing expressions of TIMP-1 and TIMP-2 genes. Furthermore, it is confirmed that the chromenens according to the present invention treats to dermal fibroblasts irradiated with UV-A, thereby stimulating an expression of a collagen synthesis marker such as procollagen and Type I collagen.

According to another aspect of the present invention, it is confirmed that the chromenes have activity of inhibiting collagenases, elastase, and matrix metalloproteinases in dermal fibroblasts. Specifically, the chromenes stimulate expressions of collagen and elastin by controlling activities of the decomposition enzymes.

Meanwhile, since it is known that hyaluronan controls a cell proliferation, an effect of UV-A on expressing hyaluronan synthetase is tested. When irradiating UV-A to cultured human dermal fibroblasts, most of HAS1, HAS2, and HAS3 mRNA are expressed and increased. An accumulation increase of hyaluronan in a culture medium is proportional to increases induced by UV-A in HAS1, HAS2, and HAS3 mRNA levels. It is considered that homeostasis in the UV-A-irradiated fibroblasts is maintained by stimulating a production of hyaluronan through up-regulations of HAS1, HAS2, and HAS3 genes. Furthermore, the present inventors confirmed that the transcriptions of AP-1 and TGF-β/Smad signaling cascade induced by UV-A is controlled by treating chromenes in the UV-A-irradiated dermal fibroblasts.

According to another aspect of the present invention, the chromenes can be obtained from an organic solvent fraction of methanol crude extract of Sargassum horneri.

According to another aspect of the present invention, the chromenes may be preferably sargachromenol D.

According to another aspect of the present invention, the photoaging protective composition may be used as cosmetic products for improving and preventing skin wrinkles, but the present invention is not limited thereto.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a mimetic diagram illustrating a process for obtaining extracts and fractions from Sargassum horneri;

FIG. 2 is a mimetic diagram illustrating a process for isolating three chromenes from 85% aq. MeOH fraction of Sargassum horneri;

FIG. 3 is a graph illustrating viability of dermal fibroblasts measured after exposed to different dose of UV-A irradiation (2 to 10 J/cm²);

FIG. 4 is a photograph illustrating morphology of dermal fibroblasts according to dose of UV-A irradiation;

FIG. 5 is a graph illustrating a hourly concentration change of TNF-α, a cytokine secretion in dermal fibroblasts exposed to UV-A irradiation through ELISA assay;

FIG. 6 is a graph illustrating a hourly concentration change of IL-1β, a cytokine secretion in dermal fibroblasts exposed to UV-A irradiation through ELISA assay;

FIG. 7 is a graph illustrating a hourly concentration change of IL-6, a cytokine secretion in dermal fibroblasts exposed to UV-A irradiation through ELISA assay;

FIG. 8 is a photograph illustrating hourly concentration changes of MMP-1, MMP-2, MMP-9, TNF-α, IL-1β, and IL-6, that are changes in MMPs and cytokine gene expression in dermal fibroblasts exposed to UV-A irradiation through a western blot assay;

FIG. 9 is a graph illustrating a result of testing effects of chromenes on viability of human dermal fibroblasts through a MTT assay;

FIG. 10 is a graph illustrating a result of chromenes on viability of HDF cells exposed to UV-A irradiation through a MTT assay (B1: No UV-A exposure, Con: Only UV-A exposure, the contents to be described below are one and the same);

FIG. 11 is a graph illustrating effects of chromenes with various concentrations on the photoaging of HDF cells that are pre-incubated with various concentrations of chromenes before exposed to UV-A irradiation;

FIG. 12 is a graph illustrating an effect of compound A on intracellular ROS generation induced by UV-A irradiation through measuring using fluorescence intensity of DCF-DA after treating with the compound A having various concentrations;

FIG. 13 is a graph illustrating an effect of compound A on intracellular ROS generation induced by UV-A irradiation through measuring using fluorescence intensity of DCF-DA after treating with the compound B having various concentrations;

FIG. 14 is a graph illustrating an effect of compound A on intracellular ROS generation induced by UV-A irradiation through measuring using fluorescence intensity of DCF-DA after treating with the compound C having various concentrations;

FIG. 15 is a graph illustrating protective effect of chromenes on membrane protein oxidation after exposed to UV-A irradiation in dermal fibroblasts;

FIG. 16 is a graph illustrating effects of compounds of the present invention on membrane lipid peroxidation induced by UV-A irradiation using diphenyl-1-pyrenylphosphine (DPPP) fluorescence probe;

FIG. 17 is a graph illustrating cellular GSH level determined using mBBR as a thiol-staining reagent according to the method described in the text measuring mBBr-GSH fluorescence intensity at λ_{excitation}=360 nm and λₑₘᵢₛₛᵢₒₙ=465 nm;

FIG. 18 is a graph illustrating effects of the compounds according to the present invention on TNF-α production in UV-A irradiated HDF cells;

FIG. 19 is a graph illustrating effect of the compounds according to the present invention on IL-1β production in UV-A irradiated HDF cells;

FIG. 20 is a graph illustrating effect of the compounds according to the present invention on IL-6 production in UV-A irradiated HDF cells;

FIG. 21 is a graph illustrating effect of compounds on mRNA expression levels of TNF-α, IL-1β, and IL-6 in dermal fibroblasts exposed to UV-A irradiation;

FIG. 22 is photographs illustrating effect of the compounds according to the present invention on inhibition of cytokines expression such as TNF-α, IL-1β, and IL-6 in dermal fibroblasts exposed to UV-A irradiation (RA: retinoic acid);

FIG. 23 is a graph illustrating time course of changes on hyaluronan secretion in UV-A irradiated cultured fibroblasts;

FIG. 24 is a graph illustrating effect of the compounds according to the present invention on UV-A induced hyaluronan secretion in cultured fibroblasts;

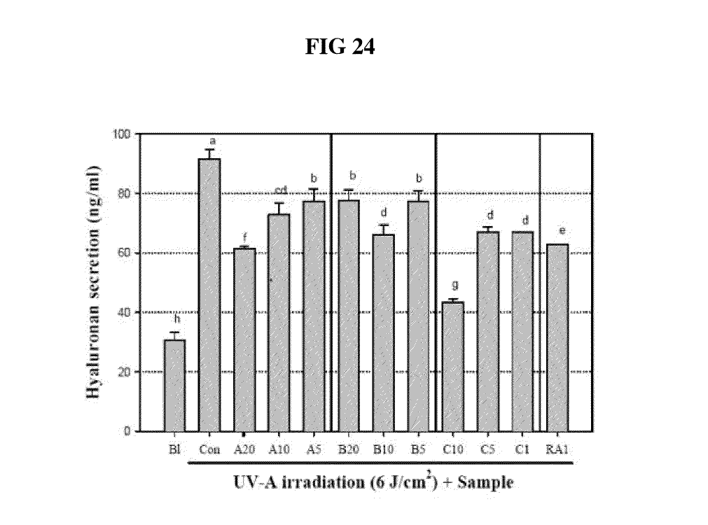

FIG. 25 is a graph illustrating effect of the compounds according to the present invention on mRNA expression levels of HAS1, HAS2, and HAS3 in UV-A irradiated dermal fibroblasts;

FIG. 26 is a graph illustrating effect of the compounds according to the present invention on collagen degrading MMP-1 secretion in human dermal fibroblasts exposed to UV-A irradiation;

FIG. 27 is a graph illustrating effect of the compounds according to the present invention on collagen degrading MMP-13 secretion in human dermal fibroblasts exposed to UV-A irradiation;

FIG. 28 is a graph illustrating effect of the compounds according to the present invention on collagen synthesis in human dermal fibroblasts exposed to UV-A irradiation;

FIG. 29 is a graph illustrating effect of the compound according to the present invention on mRNA expression levels of MMP-1, MMP-2, MMP-3, and MMP-9 in dermal fibroblasts stimulated with UV-A;

FIG. 30 is a graph illustrating effect of the compounds according to the present invention on mRNA expression levels of TIMP-1 and TIMP-2 in dermal fibroblasts stimulated with UV-A;

FIG. 31 is a graph illustrating effect of the compounds according to the present invention on protein expressions of MMP-1, MMP-2, and MMP-9 in dermal fibroblasts stimulated with UV-A;

FIG. 32 is a graph illustrating effect of the compounds according to the present invention on protein expressions of TIMP-1 and TIMP-2 in dermal fibroblasts stimulated with UV-A;

FIG. 33 is a graph illustrating effect of the compounds according to the present invention on mRNA expression levels of elastin and collagen in dermal fibroblasts stimulated with UV-A;

FIG. 34 is a graph illustrating effect of the compounds according to the present invention on protein expressions of procollagen, Type I collagen, and elastin in dermal fibroblasts stimulated with UV-A;

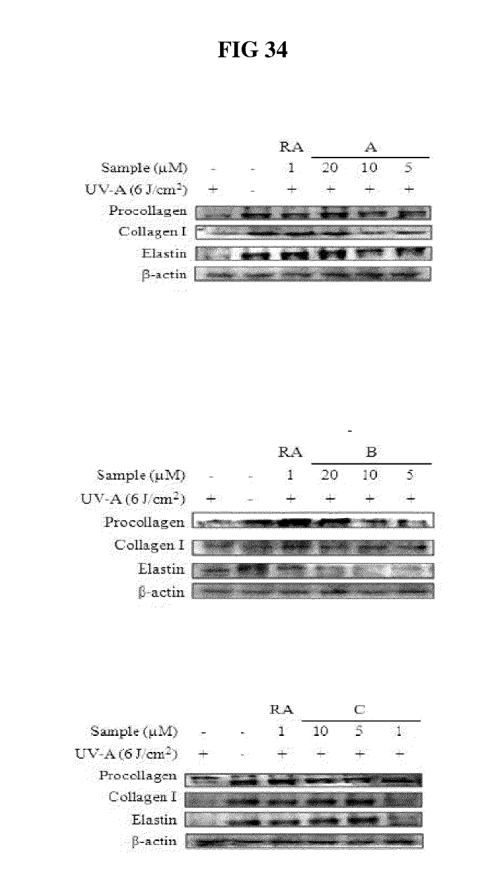

FIG. 35 is a graph illustrating effect the compounds according to the present invention at a concentration of 10 μM on collagen decomposition through a difference between UV-A non-irradiated (Blank) and UV-A irradiated cells (Control);

FIG. 36 is a graph illustrating effect of the compounds according to the present invention at a concentration of 10 μM on extracellular matrix components deposited by UV-A;

FIG. 37 is a graph illustrating effect of the compounds according to the present invention at a concentration of 10 μM on hyaluronan synthetase 1 in dermal fibroblasts exposed to UV-A irradiation;

FIG. 38 illustrates effects of the compounds according to the present invention on AP-1 signaling cascade in UV-A irradiated fibroblasts;

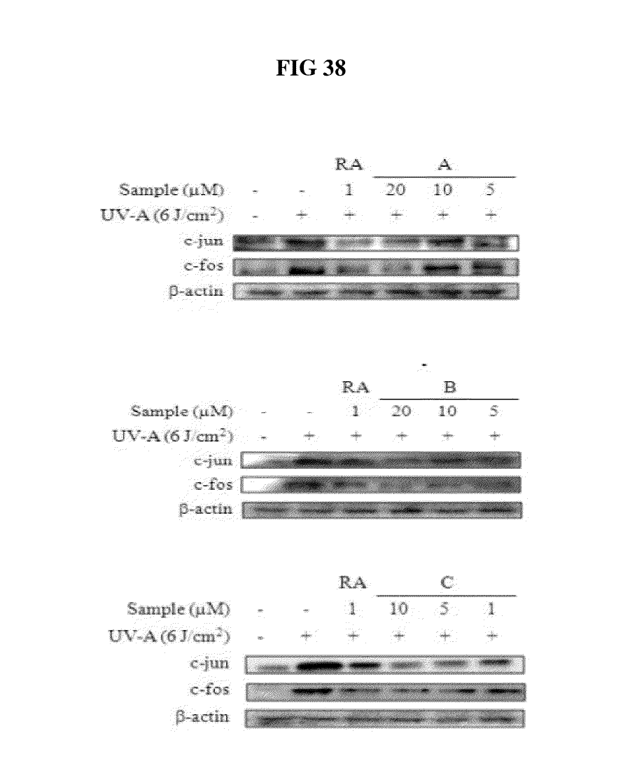

FIG. 39 is a graph illustrating effects of the compounds according to the present invention on TGF-β/Smad signaling cascade in UV-A irradiated fibroblasts; and

FIG. 40 is a graph illustrating effects of chromenes on AP-1 (A) and TGF-β/Smad (B) signaling compared to UV-A non treated group and UV-A treated group.

### DETAILED DESCRIPTION OF THE PREFERRED EMBODIMENT

All the technical terms used for the present invention have the following definitions, as long as the terms are differently defined, and have the same meanings as terms that are generally understood by those skilled in the art. In addition, a preferable method and sample are disclosed in the present specification, but things that are slimier or equal to the same are included in the range of the present invention. The contents of all the publications disclosed in the present specification as a reference are introduced in the present invention.

The term, “extract” includes conceptions including all of the extract solutions, factions, and purified materials obtained from all the stages in extracting, fractioning, or purifying, or diluent solutions, concentration solutions, and dried materials thereof.

The term, “approximately” means an amount, a level, a value, a number, a frequency, a percentage, dimension, size, weight, and length that are changed to the degree of 30, 25, 20, 25, 10, 9, 8, 7, 6, 5, 4, 3, 2, or 1% with respect to a reference amount, level, value, number, frequency, percentage, dimension, size, weight, and length.

Unless it is not differently needed in the context through the present specification, the expressions such as “include” and “including” include suggested steps or components, or the groups of steps or components, but it should be understood that they do not exclude other arbitrary steps or components, or the groups of other arbitrary steps or components.

Hereinafter, the present invention will be described in detail.

Skin Cells Photoaging Protective Composition

An example of the present invention is a photoaging protective composition including chromenes isolated from Sargassum horneri as an effective component. More specifically, three chromenes, such as sargachromenol (Compound A), sargachromenol E (Compound B), and sargachromenol D (Compound C) were isolated from 85% aq. MeOH fraction of Sargassum horneri, brown algae.

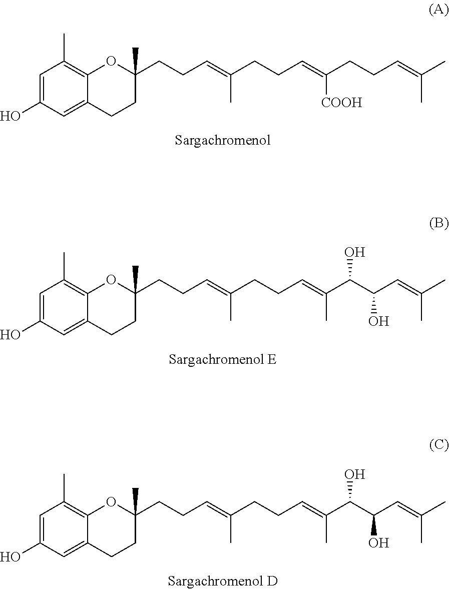 [SMILES: CC(C)=CCC/C(=C/CC/C(C)=C/CC[C@]1(C)CCc2cc(O)cc(C)c2O1)C(=O)O ; CC(C)=C[C@H](O)[C@@H](O)/C(C)=C/CC/C(C)=C/CC[C@]1(C)CCc2cc(O)cc(C)c2O1 ; CC(C)=C[C@@H](O)[C@@H](O)/C(C)=C/CC/C(C)=C/CC[C@]1(C)CCc2cc(O)cc(C)c2O1]

Method for Extracting Chromenes

Water or an organic solvent may be used as a proper solvent for the extraction, and preferably may be alcohols having a carbon number of 1 to 4, such as methanol, ethanol, propanol, isopropanol, and butanol, all sorts of solvents such as acetone, ether, benzene, chloroform, ethyl acetate methylene chloride, dichloromethane, hexane, and cyclohexane, or mixed solvent thereof.

In addition, the extract that is extracted by using the above-described solvents may be further fractionated by using a solvent selected from the group consisting of hexane, butanol, dichloromethane, acetone, ethyl acetate, ethyl ether, chloroform, water, and a mixture thereof. Preferably, the above-described solvent may be dichloromethane, ethyl acetate, and n-butanol. The solvent at the time of fractionating may be in at least one solvent. The extract according to the present invention extracted from Sargassum horneri may be prepared according to a general method of preparing marine algae extracts, specifically, a macerating extraction method, a digesting extraction method, or a hydrothermal extraction method, and may be prepared by using a general extraction apparatus, ultrasonicator, or homogenizer. In addition, since then, the solvents in the extract extracted by using the solvents described above may be removed through performing a filtration process, a concentration process, or drying process, and also performing all the filtration, concentration, and drying processes. Specifically, the filtration process may use a filter paper or a decompression filter, the concentration process may be performed by using a vacuum concentrator, for example, a rotary evaporator, and the drying process may be performed by using a freeze drying process, for example. Furthermore, the Sargassum horneri extract obtained may be stored in a deep freezer until it is used, water in the extract may be completely removed through a concentration and freeze-drying process. The Sargassum horneri extract without water may be used in a type of powder, or may be used through dissolving the powder in distilled water or a general solvent.

As an example of the present invention, the Sargassum horneri extract may be prepared by removing salt and impurities in Sargassum horneri, drying to prepare a dried sample, adding to the dried sample to prepare a crude extraction liquid, and then vacuum-concentrating the crude extraction liquid. At this time, the removing of salt and impurities in Sargassum horneri may be performed by washing it with running water and the dried sample may be prepared by drying the Sargassum horneri without the salt and impurities, and then grinding the dried Sargassum horneri. Furthermore, the dried Sargassum horneri sample may be stored in a deep freezer until it is used. In addition, water in the Sargassum horneri extract may be completely removed through concentrating and freeze-drying the obtained extraction liquid, and the Sargassum horneri extract without water may be used in a type of powder or by dissolving it in distilled water or a general solvent.

Effectiveness of Chromenes

Number of studies reported that UV radiation decreases collagen synthesis and increases the levels of collagen degrading MMPs expression in dermal fibroblasts. Thus, less synthesis and increased degradation of collagen by collagenase MMPs are assumed to be involved in wrinkle formation and collagen deficiency in photodamaged skin. Retinoic acid is well known as a potential anti-aging drug. However, general use of retinoic acid has limitation due to its skin irritancy and mutagenicity. Therefore, in the present invention, three chromenes were isolated from Sargassum horneri. Effect of chromenes on the regulation of collagen synthesis and collagen degradation by MMPs in UV-A irradiated human dermal fibroblasts (HDF) were investigated to find out natural anti-aging agent from marine resources.

Exposure of skin to the UV light results in the activation of reactive oxygen species (ROS) system. ROS include activated and/or free radical oxygen compounds such as singlet oxygen, superoxide anion, hydroxyl radical and hydrogen peroxide. These species can attack the tissues in the dermis or epidermis to cause skin aging. Therefore, free radical scavenging compounds could also be used as a cosmetic ingredient to relieve the skin aging. As a result, in the present invention, effects of chromenes on free radical scavenging and dermal fibroblasts damage through the oxidation of membrane lipid and protein were evaluated followed by several antioxidant assays. The results indicated that three chromenes reduced ROS generation, membrane lipid peroxidation and protein oxidation caused by UV-A irradiation.

In wrinkle formation, UV-A irradiation is closely related with collagen degradation of skin dermis by activating the matrix degrading enzymes. Matrix metalloproteinases (MMPs) are a family of zinc-dependent endopeptidases that are secreted as inactive zymogens (pro-MMPs) requiring extracellular activation. These MMPs have ability to collectively degrading all components of the extracellular matrix and their activation is strictly regulated by specific tissue inhibitors of MMPs (TIMPs). Among MMPs, many studies have been focused on the gelatinases and collagenases which are considered to be involved in photoaging in relation to wrinkle formation.

As a main MMP groups, MMP-2 (gelatinase A or 72 kDa type IV collagenase) and MMP-9 (gelatinase B or 92 kDa type IV collagenase) are the primary matrix-degrading enzymes produced by fibroblasts. They are able to degrade type IV, V, VII and X collagens, elastin and denatured collagens in dermis of skin. In relation to collagen deficiency, collagenases are capable of degrading native collagen fibers, which are the major components of the connective tissues. Collagenase MMPs cleave collagen type I, II and III at a specific region generating three-fourths N-terminal and one-fourth C-terminal fragments and become susceptible to degrading by other type of MMPs, such as gelatinases. Overexpression of gelatinases and collagenases by UV irradiation may result in collagen deficiency in human conjunctivochalasis fibroblasts. The results demonstrated that the UV-A irradiation up-regulated collagenases and gelatinases expression such as MMP-1, MMP-2, MMP-3 and MMP-9 genes whereas these expression levels were decreased by chromenes treatment in UV-A irradiated dermal fibroblasts. Moreover, it was observed that these MMPs expressions were inhibited by TIMP-1 and TIMP-2 genes.

Additionally, elastin is an extracellular matrix protein providing elasticity to the connective tissues. It forms elastic fiber in the skin dermis and has an influence on skin elasticity. Damage to the elastin fibers leads to the decreased skin elasticity. Elastase is the proteinase enzyme capable of degrading elastin. Therefore, inhibition of the elastase activity could be used as a method to protect against skin aging. The results showed that chromenes inhibited human fibroblast elastase activity.

Meanwhile, hyaluronan is also major component of skin and it has vital roles in cell adhesion, migration and differentiation mediated via various hyaluronan-binding proteins. A number of functions have been described to low-molecular weight hyaluronan in inflammatory processes. In particular, an increased content of low molecular-weight hyaluronan has been found in chronically inflamed tissue. Thus, hyaluronan of reduced molecular weight, either due to synthesis of lower molecular-weight compound or fragmentation of existing hyaluronan, has been regarded as an important component to the mechanism of inflammation and subsequent tissue degradation. Cytokines are known to interact to regulate the molecular weight as well as the quantity of hyaluronan.

Recently, three human hyaluronan synthase (HAS1, HAS2 and HAS3) genes have been identified and their features elucidated. These enzymes have been cloned and characterized as being regulated by inflammatory cytokines. It was also demonstrated that hyaluronan synthase 1 and 2 synthesizes high molecular-weight of hyaluronan, whereas hyaluronan synthase 3 generates low molecular-weight hyaluronan. Thus, the overall processes as well as individual sequences of hyaluronan biosynthesis have been identified. This study investigated the effect of chromenes on the expression of hyaluronan synthase mRNA in UV-A irradiated cultured dermal fibroblasts, and explored the cytokine regulation under inflammatory conditions stimulated by 6 J/cm2 of UV-A. In this study, we show that the mRNA expression of hyaluronan synthase 1, 2 and 3 were remarkably enhanced by UV-A irradiation after 24 h followed by TNF-α and IL-1β expression were increased moderately by UV-A irradiation. It has been reported that reactive oxygen species have been shown to depolymerise hyaluronan. In addition, a number of mammalian hyaluronidases capable of enzymatically degrading hyaluronan have recently been cloned and shown to be associated with inflammation. Thus, the accumulation of hyaluronan might result from mechanisms such as depolymerisation and enzymatic cleavage by UV-A irradiation, in addition to synthesis of hyaluronan by increment of hyaluronan synthases expression. Thus, hyaluronan produced by hyaluronan synthases, in addition to the destructive effects of reactive oxygen species and cytokines might synergistically potentiate the destruction of the skin components. These effects were negatively regulated by chromenes treatment.

Next, we evaluated whether chromenes can affect the elastic properties, the levels of procollagen, collagen type I and elastin were examined as synthetic markers of elastic fibers. The collagen molecules are synthesized in osteoblasts and fibroblasts, secreted into the extracellular space as procollagens. During the secretion, aminoterminal and carboxyterminal propeptides are cleaved from the procollagen molecule by specific proteases. There are 29 types of collagen have been identified, but over 90% of the collagen in the body is type I. Especially, collagen type I is known to major components of skin dermis and belongs to a group of proteins supplying tensile strength and stability.

Of all the constitutive components of the basement membrane of skin such as proteoglycans, entactin, laminin, fibronectin and collagen are responsible for stability of basement membranes. Especially, loss of collagen and fibronectin by UV-irradiation may result in change of basement membranes at the dermal-epidermal junction. Treatment with chromenes decreased loss of collagen I by increment of collagen synthesis and inhibition of fibronectin degradation. Our results revealed that the presence of chromenes in UV-A irradiated dermal fibroblasts attenuated components of the basement membrane degradation through the transcriptional regulation of AP-1 and TGF-β/Smad signaling pathway. These activations depend on the availability of particular chemicals that are capable of reacting with the specific kinds of functional groups that exist in chromenes. Therefore, the composition and structure of compounds must be considered to be useful as a direct focus of study.

Chromenes related with the tocotrienol are frequently found among the brown algae, particularly abundant within the Sargassum genus. Tocotrienols are fat-soluble molecules related to the family of tocopherols. Structurally, tocopherols and tocotrienols share some resemblance consisting of a common chromanol head and a side chain at the C-2 position. However, tocopherols and tocotrienols are distinguished by their side chains. While tocopherol has a saturated phytyl tail, tocotrienol possesses an unsaturated isoprenoid side chain. Tocotrienols have been shown to have unique functional properties. Interestingly, tocotrienols have been shown to reduce plasma cholesterol levels as well as other lipid and non-lipid related risk factors for cardiovascular disease.

The hydroxyl group (phenolic) is necessary for chromenes to function as antioxidant activities. The phenolic hydroxyl group on the chromanol ring of chromenes reacts with free radical, causing termination of the auto-oxidation chain reaction. This action is called free-radical scavenging and involves donation of the phenolic hydrogen to a fatty acid free radical as well as free radical quenching. In this process, an inactive tocopheroxyl free radical is formed, which is converted to tocopherylquinone. The quinone form is reduced back to the tocopherol form by reducing agent such as ascorbic acid, thus chromene molecules are recycled and might be repeatedly function as antoxidants.

Sargachromenol has carboxyl group in the molecule. Deprotonation of a carboxylic acid gives a carboxylate anion, which is resonance stabilized because the negative charge is delocalized between the two oxygen atoms increasing its stability. Each of the carbon-oxygen bonds in a carboxylate anion has partial double-bond character. Sargachromenol E and Sargachromenol D have diol group in their isoprenoid side chain. We assume that deprotonation of hydroxyl groups of chromenes molecules might have contributed to chelation of zinc metal ion, hence, the levels of collagen degrading MMPs expression by UV irradiation were inhibited in presence of chromenes.

Therefore, these results suggest that increase of collagen degrading MMPs activities by UV exposure might contribute to wrinkle formation through decomposition of basement membranes at the dermal-epidermal junction thereby degradation of extracellular matrix components such as elastic fibers. Therefore, it can be suggested that topical application of chromenes might be an effective strategy to prevent wrinkle formation caused by UV irradiation.

The following Table 1 is summarized test results relating to effects of three chromenes according to the present invention on preventing and treating UV-induced skin aging:

TABLE 1

| | | | | 1 μM |
|---|---|---|---|---|
| | Samples (10 μM) | | | Retinoic |
| | A | B | C | acid |
| Fibroblasts protection (%) | 37.25 | 17.38 | 26.30 | 12.21 |
| ROS scavenging (%) | 38.58 | 11.87 | 25.82 | 46.60 |
| Inhibition of protein | 30.57 | 15.27 | 60.11 | 55.36 |
| oxidation (%) | | | | |
| Inhibition of lipid | 15.63 | 3.13 | 18.75 | 21.56 |
| peroxidation (%) | | | | |
| Enhancement of GSH level (%) | 23.17 | 19.51 | 24.39 | 15.85 |
| Inhibition of TNF-α secretion (%) | 20.59 | 8.79 | 28.82 | 24.42 |
| Inhibition of IL-1β secretion (%) | 10.07 | 8.87 | 9.83 | 6.71 |
| Inhibition of IL-6 secretion (%) | 11.11 | 8.89 | 18.52 | 14.07 |
| Hyaluronan (%) | 16.67 | 13.33 | 52.22 | 31.11 |
| Elastase inhibition IC₅₀ (μM) | 11.28 | 13.77 | 6.78 | 1.04 |
| MMP-1 inhibition (%) | 27.76 | 19.96 | 40.45 | 30.69 |
| MMP-13 inhibition (%) | 23.82 | 28.61 | 38.15 | 37.14 |
| Procollagen (%) | 115.79 | 97.37 | 123.68 | 57.89 |

Cosmetic Products for Improving Skin Wrinkles

The cosmetic products for improving skin wrinkles according to the present invention may include chromenes selected from Sargachromenol E, Sargachromenol D, and a mixture thereof, derived from Sargassum horneri as an effective component, and may be used in combination with a dermatologically acceptable excipient.

The cosmetic products for improving wrinkles of the present invention may be formulated into a cosmetic product, such as a nutrition tonor, a skin softner, a nutrition cream, a massage cream, a pack, a gel, an astringent, an essence, an eye cream, an eye essence, a cleansing cream, a cleansing form, a cleansing water, a powder, a body lotion, a body cream, a body oil, and a body essence, but the present invention is not limited thereto.

An example of the excipient may include a skin softner, a skin penetration enhancer, a coloring agent, a flavoring agent, an emulsifying agent, a thickening agent, and a solvent, but the present invention is not limited thereto. In addition, it may further include a perfume, a pigment, a germicide, an antioxidant, a preservative, humectants, and the like, and also a viscosity increasing agent, inorganic salts, a synthetic polymer, and the like for improving physical properties.

For example, in a case where a cleanser and soap including the chromenes of the present invention is prepared, it can be easily prepared by adding the chromenes to a general cleanser and soap base. In a case of preparing a cream, it can be prepared by adding the chromenes or salts thereof to a general oil in water (O/W) cream base. Here, a synthetic or natural material such as proteins, minerals, and vitamins for improving the physical properties, for example, a perfume, a chelating agent, a pigment, an antioxidant, a preservative, and the like.

The content of sargachromenol included in the cosmetic product for improving skin wrinkles according to the present invention may be included in an amount range of 0.0001 to 50 wt %, and preferably 0.001 to 10 wt % relative to the total weight of the cosmetic product. The reason is that when in is less than 0.001 wt %, the effect cannot be expected, and when it exceeds 10 wt %, safety cannot be obtained and dosage form cannot be formulated. In addition, in order to more effectively improve skin wrinkles and improve a formulation ability of dosage form, it is more preferable that the content be used in an amount range of 0.01 to 5 wt % relative to the total weight of the composition.

In addition, the cosmetic product for improving skin wrinkles may preferably include other components for improving wrinkles that can give a synergic effect to the effect on improving wrinkles in the range of not damaging the effect for improving wrinkles that is an abject of the present invention in addition to the chromenes derived from Sargassum horneri.

Hereinafter, the present invention will now further be described with reference to the accompanying Examples. Examples are only for illustrating specifically the present invention, but the range of the present invention is not limited to Examples.

### Abbreviation

Compound A: Sargachromanol
Compound B: Sargachromanol E
Compound C: Sargachromanol D

### Example 1

### Sargassum horneri Extract

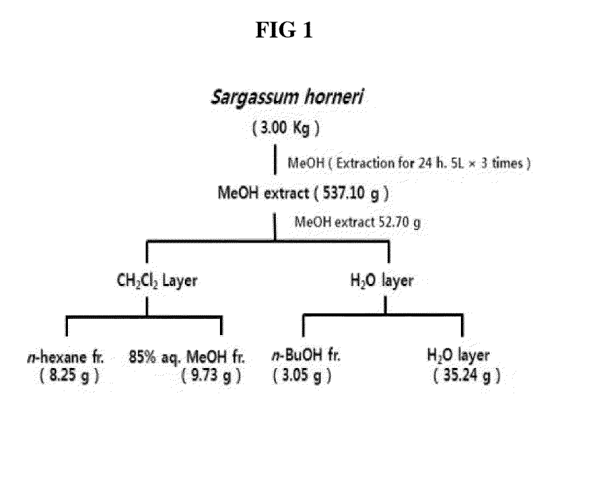

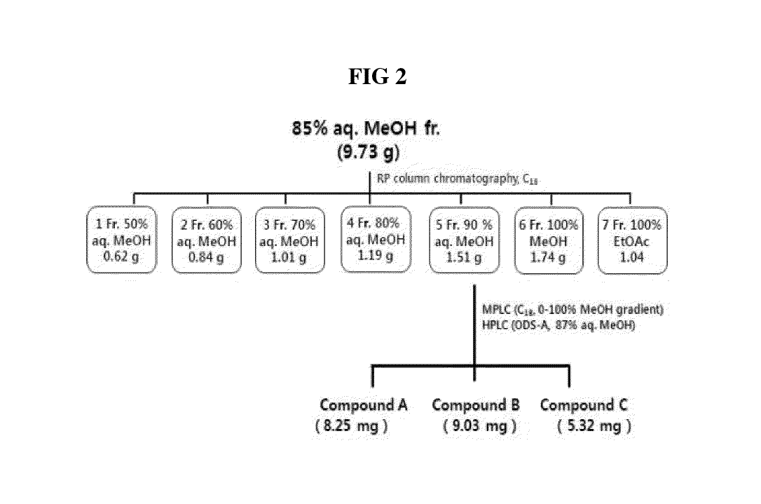

The brown alga Sargassum horneri was purchased from para jeju Co. in 2010. The samples were briefly dried under shade, and then samples of S. horneri were ground to a powder and extracted for 3 times with MeOH. The crude extracts (500 g) were distributed in 2 L of water; 2 L of dichloromethane (CH₂Cl₂) was added thereto; and partitioned between CH₂Cl₂ and water (repeated for 3 times). Sin then, the layer of dichloromethane was concentrated to further partition between 85% aq. MeOH (9.73 g) and n-hexane (8.25 g), and the aqueous layer was fractionated with n-BuOH (3.05 g) and H₂O (35.24 g) by adding the same amount of n-BuOH (see FIG. 1). Among them, the 85% aqueous MeOH fraction was subjected to C₁₈ reversed-phase vacuum flash chromatography using stepwise gradient mixtures of MeOH and water (50, 60, 70, 80, 90% aq. MeOH and 100% MeOH), and finally 100% ethyl acetate as eluent to give 7 subfractions. Based on the reported data, the moderately polar fractions were separated. Therefore, the fifth fraction (1.506 g) was subjected to reversed-phase Medium Pressure Liquid Chromatography, MPLC (0-100% MeOH gradient) to yield 15 fractions. Then subjected to HPLC (YMC ODS-A, 87% aq. MeOH, 1 cm×25 cm, 3 ml/min). Subfractions 7 and 9 were identified as compound B (9.03 mg) and C (5.32 mg). Purification of fraction 14 by semi-preparative C₁₈ HPLC (YMC ODS-A column, 83% aq. MeOH, 0.46 cm×25 cm, 1 ml/min) gave compounds A (8.25 mg) (see FIG. 2).

Test Method

1. Cell Culture

Chito oligosaccharides (COS) of 3 to 5 kDa were obtained from Kitto Life Co. (Seoul, Korea). Human dermal fibroblasts (HDF cells) were purchased from Promo cell (Heidelberg, Germany) which were isolated from the dermis of adult skin.

HDF cells were grown in Dulbecco's modified Eagle's medium (DMEM, Gibco-BRL, Gaithersburg, Md. USA) containing 10% FBS, 2 mM glutamine and 100 μg/ml penicillin-streptomycin (Gibco-BRL, Gaithersburg, Md., USA) at 37° C. humidified atmosphere of 5% CO₂. HDF Cells (passage 2) were maintained for 6 additional passages and sub-cultured for experiment at about 90 to 95% confluence by detaching with trypsin-EDTA solution.

2. UV-A Irradiation

HDF cells were seeded in 24-well plate at a density of 1×10⁵ cells/well with DMEM containing 10% FBS, 2 mM gluramine and 100 μg/ml penicillin-streptomycin at 37° C. humidified atmosphere of 5% CO₂. After incubation for 24 h, the cells were exposed to UV-A energy at the range of 6 J/cm² (365 nm UV-A light source, Bio-Sun lamp, Vilber Lourmat, Marine, France) in 200 μl of phosphate buffered saline (PBS) to each well. After UV-A irradiation, the cells were incubated for 24 h in serum-free DMEM.

3. MTT Assay

Viability level of HDF cell was determined through ability of mitochondria capable of converting 3-(4,5-dimethylthiazol-2-yl)-2,5-diphenyl tetrazolium bromide (MTT) into a insoluble formazan product. Dermal fibroblasts were grown in 96-well plates at a density of 1×10⁴ cells/well. After 24 h, after irradiating UV-A (6 J/cm²) to the cell, the cells were cultured with the chromenes sample or without the chromenes sample at 37° C. humidified atmosphere of 5% CO₂. The supernatant medium was removed, and the cells were incubated with 100 μl of MTT (1 mg/ml) for 4 h. The unconvertived MTT was removed, dimethyl sulfoxide (DMSO) was added, and then cell viability was measured at the absorbance at 540 nm using a microplate reader (Tecan Austria GmbH, Austria) to determine the amount of formazan in a cell. Relative cell viability was quantified as a percentage compared to the control group.

4. Phototoxicity Determination

HDF cells are exposed to UV-A light in the presence of chromenes and retinoic acid in concentrations of 5, 10, and 20 μM and 1, 2, and 5 μM, respectively. After 24 h, cell viability was determined by MTT assay.

5. Effect of Chromenes on Elimination Ability of Intracellular Reactive Oxygen Species (ROS) in a Cell Irradiated with UV-A

Intracellular formation of reactive oxygen species (ROS) was assessed using oxidation sensitive dye 2′,7′-dichlorofluorescin diacetate (DCFH-DA) as the substrate. HDF cells growing in fluorescence microtiter 96-well plates for 24 h were exposed to UV-A (6 J/cm²). The exposed cells were loaded with 20 μM DCFH-DA dissolved in PBS and incubated for 30 min in the dark at 37° C. humidified atmosphere of 5% CO₂. Finally, after washing the cells with PBS for two times. The formation of 2′,7′-dichlorofluorescein (DCF) dissolved in PBS due to oxidation of DCFH in the presence of various ROS was read after every 20 min at the excitation wavelength (Ex) of 485 nm and the emission wavelength (Em) of 535 nm using a fluorescence microplate reader (Tecan Austria GmbH, Salzburg, Austria).

6. Cell Membrane Protein Damage Caused by UV-A Irradiation

The amount of carbonyl groups in cell membrane proteins was determined to assess the cellular protein oxidation level that is a standard of determining the degree of cell membrane damage. HDF cells were co-cultured with various concentrations of compounds for 24 h after 6 J/cm² of UV-A irradiation. Cell were washed three times with PBS and lysed in lysis buffer without reducing agents (25 mM Tris-HCl, pH 7.8, 2 mM EDTA, 180 mM NaCl and 1% Triton X-100). Aliquots of cell lysates were separately incubated then trichloroacetic acid (400 μl from 20% trichloroacetic acid) was added to each reaction mixture and solubilized proteins were precipitated by centrifugation at 3,000 rpm for 15 minutes. Precipitated protein was resuspended in 2% 2,4-dinitrophenylhydrazine (2 mM HCl) and incubated at room temperature for 20 min. The protein was precipitated again with 20% trichloroacetic acid and the pellet was washed three times with ethanol:ethylacetate (1:1 v/v) solution. The pellet was then dissolved in 6 M guanidin hydrochloride (500 μl) and incubated for 15 min at 37° C. After centrifugation at 1,500 g for 5 min, absorbance of the supernatant was recorded against a complementary blank at 370 nm using a GENios® fluorescence microplate reader (Tecan Austria GmbH, Salzburg, Austria).

7. Determination of Cell Membrane Lipid Peroxidation

Intracellular lipid hydroperoxide levels were determined by the fluorescence probe, diphenyl-1-pyrenylphosphine (DPPP). HDF cells were seeded into fluorescence microtiter 96-well plates at a density of 1×10⁸ cells/ml using serum-free media. After 80% confluence, HDF cells were treated with various concentrations of compounds and incubated for 24 h after 6 J/cm² of UV-A irradiation. Then, cells were washed three times with PBS and labeled with 13 μM DPPP (dissolved in DMSO) for 24 h at 37° C. in the dark. DPPP oxide fluorescence intensity was measured after 24 h at the excitation wavelength (Ex) of 361 nm and the emission wavelength (Em) of 380 nm using a GENios® fluorescence microplate reader (Tecan Austria GmbH, Salzburg, Austria).

8. Measurement of Intracellular GSH

Intracellular glutathione (GSH) level in intact cells was determined using monobromobimane as a thiol-staining reagent. HDF cells were seeded into fluorescence microtiter 96-well plates at a density of 5×10³ cells/well. Cell were washed three times with PBS and treated with various concentrations of the compounds after 6 J/cm² of UV-A irradiation and incubated for 30 min. 0.04 mM of Monobromobimane was added to cells and staining was carried out for 24 h at 37° C. in the dark. After staining fluorescence intensity was measured (excitation and emission: 360 and 465 nm) using a GENios® fluorescence microplate reader (Tecan Austria GmbH, Salzburg, Austria).

9. Measurement of MMP, Cytokines (TNF-α, IL-1β, IL-6), Hyaluronan, and Collagen Concentrations by Enzyme-Linked Immunosorbent (ELISA) Assay

For measuring various collagen degradation MMP and inflammatory cytokine, collagen content in a cell, an enzyme-linked immunosorbent assay was used after UV-A irradiation. After irradiating HDF cell with UV-A, the cells were incubated with various concentrations of sample. After 24 h, the supernatants of the HDF cells were collected, and used for testing MMP, cytokine, hyaluronan, and collagen. For testing, Biotrak™ ELISA kits (Amersham Pharmacia Biosciences, England, UK), procollagen ELISA kit (Takara Bio Inc), and Hyaluronan ELISA kits (R&D system) were used according to the manufacturer's instructions.

10. Measurement of Elastase Activity

Elastase activity was measured using the elastase substrate STANA (N-succinyl-(Ala)3-p-nitroanilide). For elastase enzyme extraction from the cell, cultured fibroblasts at 80% confluence were washed once with phosphate-buffered saline (PBS), scraped into PBS, and centrifuged at 4° C., 1,000 rpm for 5 min. The cell pellets were lysed with 0.1 M Tris-HCl (pH 7.6) buffer containing 0.1% Triton-X 100 followed by ultrasonication for 5 min on ice. Cleared supernatants after the removal of cell residues by centrifugation (2,000 rpm, 10 min) were used as the fibroblast enzyme solution. In brief, 100 μl of enzyme solution was dispensed into 96-well plates, which were pre-incubated for 15 min at 37° C. After addition of 2 μl of 62.5 mM STANA, the plates were further incubated for 1 h at 37° C. The release of p-nitroaniline was measured by absorbance at 410 nm and enzymatic activity is expressed as unit per mg protein, one unit representing the activity that releases 1 nmol of nitroaniline per hour.

11. Immunostaining

Immunostaining is used to determine the subcellular distribution and organization of proteins. Cells were grown in 6-well plates, Aspirate liquid, then cover cells to a depth of 2-3 mm with 4% formaldehyde in PBS for 15 min at room temperature. Aspirate fixative, rinse three times in PBS for 5 minutes each then incubated primary antibody and for overnight at 4° C. Rinse three times in PBS for 5 min each. Incubate specimen in fluorochrome-conjugated secondary antibody diluted in Antibody Dilution Buffer for 1-2 h at room temperature in dark and incubated with Hoechest 33342 (Sigma-Aldrich Co. MO, USA). For best results, examine specimens immediately using appropriate excitation wavelength. For long-term storage, store slides flat at 4° C. protected from light. The image of HDF cells were collected using Leica invented microscopy (DM IRB) with 40× objective and a digital CCD camera (CTR 6000, Leica, Wetzlar, Germany) under fluorescence.

12. Reverse Transcriptase Polymerase Chain Reaction (RT-PCR) Analysis

Total cellular RNA was isolated using Trizol reagent (Invitrogen Co., CA, USA). The 2 μg of isolated RNA was reverse transcribed into cDNA using oligo-(dT) primer (Promega, Madison, Wis., USA). Target cDNA was amplified using the forward and reverse primer sequences. The amplification was carried out at 95° C. for 45 sec, 60° C. for 50 sec and 72° C. for 60 sec for cycles. After amplification step, extension process proceeded consecutively at 72° C. for 5 min. PCR products were separated on 1.5% agarose gel for 10 min at 100 V by electrophoresis. Gels were stained with 1 mg/ml of EtBr and photographed by UV illumination using AlphaEase® gel image analysis software (Alpha Innotech., San Leandro, Calif., USA).

TABLE 2
MMPs-specific primers used for the RT-PCR

| Gene | Direction | Sequence |
|---|---|---|
| MMP-1 | Forward | 5′-GAT-GTG-GAG-TGC-CTG-ATG-TG-3′ |
| | Reverse | 5′-TGC-TTG-ACC-CTC-AGA-GAC-CT-3′ |
| MMP-2 | Forward | 5′-TGA-AGG-TCG-GTG-TGA-ACG-GA-3′ |
| | Reverse | 5′-CAT-GTA-GCC-ATG-AGG-TCC-ACC- |
| | | AC-3′ |
| MMP-3 | Forward | 5′-TCT-GCA-AGG-TTA-TCC-CAA-GG-3′ |
| | Reverse | 5′-TAT-TCC-TGG-AAA-GGC-ACC-TG-3′ |
| MMP-9 | Forward | 5′-CAC-TGT-CCA-CCC-CTC-AGA-GC-3′ |
| | Reverse | 5′-CAC-TTG- TCG-GCG-ATA-AGG-3′ |
| TIMP-1 | Forward | 5′-AAT-TCC-GAC-CTC-GTC-ATC-AG-3′ |
| | Reverse | 5′-TGC-AGT-TTT-CCA-GCA-ATG-AG-3′ |
| TIMP-2 | Forward | 5′-TGA-TCC-ACA-CAC-GTT-GGT-CT-3′ |
| | Reverse | 5′-TTT-GAG-TTG-CTT-GCA-GGA-TG-3′ |
| β-actin | Forward | 5′-GAG-TCA-ACG-GAT-TTG-GTC-GT-3′ |
| | Reverse | 5′-GAC-AAG-CTT-CCC-GTT-CTC-AG-3′ |

TABLE 3
Inflammatory Cytokines-specific primers

| Gene | Direction | Sequence |
|---|---|---|
| TNF-α | Forward | 5′-GGA-GCC-AGC-TCC-CTC-TAT-TT-3′ |
| | Reverse | 5′-GGC-TAC-ATG-GGA-ACA-GCC-TA-3′ |
| IL-1β | Forward | 5′-CTG-TCC-TGC-GTG-TTG-AAA-GA-3′ |
| | Reverse | 5′-TTC-TGC-TTG-AGA-GGT-GCT-GA-3′ |
| IL-6 | Forward | 5′-AGG-AGA-CTT-GCC-TGG-TGA-AA-3′ |
| | Reverse | 5′-CAG-GGG-TGG-TTA-TTG-CAT-CT-3′ |
| β-actin | Forward | 5′-GAG-TCA-ACG-GAT-TTG-GTC-GT-3′ |
| | Reverse | 5′-GAC-AAG-CTT-CCC-GTT-CTC-AG-3′ |

TABLE 4
Elastic gene-specific primers

| Gene | Direction | Sequence |
|---|---|---|
| Elastin | Forward | 5′-GGC-CAT-TCC-TGG-TGG-AGT-TCC-3′ |
| | Reverse | 5′-AAC-TGG-CTT-AAG-AGG-TTT-GCC- |
| | | TCC-A-3′ |
| Collagen | Forward | 5′-CAG-TCG-CTT-CAC-CTA-CAG-CA-3′ |
| I | Reverse | 5′-GGT-GGA-GGG-AGT-TTA-CAC-GA-3′ |
| TGF-β1 | Forward | 5′-CGA-GTG-CCA-AAT-GAA-GAG-GAG- |
| | | C-3′ |
| | Reverse | 5′-AAA-CCT-GAG-CCA-GAA-CCT-GAC- |
| | | G-3′ |
| β-actin | Forward | 5′-GAG-TCA-ACG-GAT-TTG-GTC-GT-3′ |
| | Reverse | 5′-GAC-AAG-CTT-CCC-GTT-CTC-AG-3′ |

TABLE 4
Hyaluronan synthases-specific primers

| Gene | Direction | Sequence |
|---|---|---|
| HAS1 | Forward | 5′-ACC-ATC-GCC-TTC-GCC-CTG-CTC- |
| | | ATC-C-3′ |
| | Reverse | 5′-CCC-GCT-CCA-CAT-TGA-AGG-CTA- |
| | | CCC-A-3′ |
| HAS2 | Forward | 5′-TTT-CTT-TAT-GTG-ACT-CAT-CTG- |
| | | TCT-CAC-CGG-3′ |
| | Reverse | 5′-ATT-GTT-GGC-TAC-CAG-TTT-ATC- |
| | | CAA-AGG-G-3′ |
| HAS3 | Forward | 5′-CAG-AAG-GCT-GGA-CAT-ATA-GAG- |
| | | GAG- GG-3′ |
| | Reverse | 5′-ATT-GTT-GGC-TAC-CAG-TTT-ATC- |
| | | CAA-ACG-3′ |
| β-actin | Forward | 5′-GAG-TCA-ACG-GAT-TTG-GTC-GT-3′ |
| | Reverse | 5′-GAC-AAG-CTT-CCC-GTT-CTC-AG-3′ |

13. Western Blot Analysis

Whole cells were lysed in RIPA buffer (Sigma-Aldrich Corp., St. Louis, USA). After centrifugation, total protein amount of cell lysates was determined using Lowry method (BioRad Laboratories, Hercules, Calif., USA). Aliquot of supernatant containing equal amounts of proteins (15 μg) was electrophoresed on 10% or 12% SDS-PAGE gels, transferred onto a nitrocellulose membrane (Amersham Pharmacia Biotech., England, UK), blocked with 5% skim milk in TBS containing 0.1% Tween 20 (TBS-T) for at least 1 h and hybridized with the primary antibodies (Santa Cruz Biotechnology Inc., CA, USA). All primary monoclonal antibodies were diluted with TBS-T at 1:1000 ratio. Bound antibodies were detected by horseradish peroxidase-conjugated secondary antibodies for 1 h at room temperature and the immunoreactive proteins were detected using a chemiluminescent ECL assay kit (Amersham Pharmacia Biosciences, England, UK) according to the manufacturer's instructions. Western blot bands were visualized using a LAS3000® Luminescent image analyzer (Fujifilm Life Science, Tokyo, Japan).

### Experimental Example 1

### Optimization of UV-A Irradiation Dosage

To determine the optimum energy level of UV-A irradiation, the cultured human dermal fibroblasts were exposed to various UV-A energy sources within a various range and the cytotoxicity was measured by MTT assay in a time dependant manner.

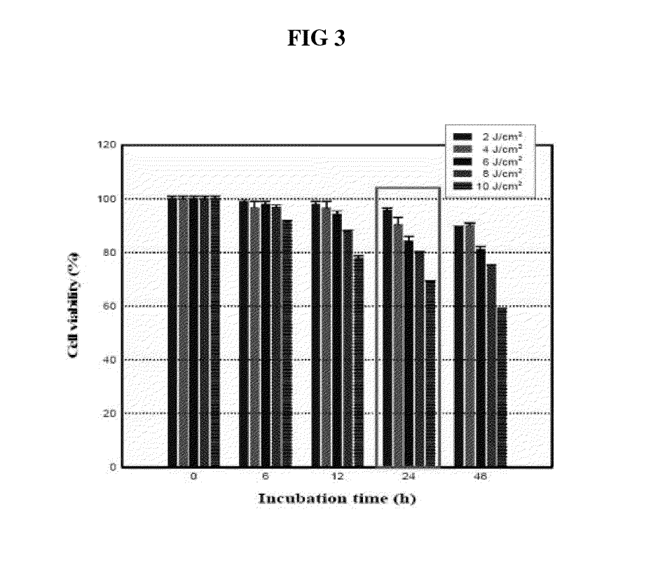

As illustrated in FIG. 3, cell viability assays showed that cells were significantly damaged after 24 h incubation followed by UV-A radiation. Therefore, incubation period for 24 h was chosen as the optimum condition. Shorter incubation than 24 h failed to show initiation of cell damage while 48 h incubation significantly caused cell loss which can produce unreliable results for further experiments. In case of UV-A irradiation dosage, cell viability assays pointed out the 6 J/cm² as the optimum energy level. Smaller dosages than 6 J/cm² did not initiate the cell damage, while 8 and 10 J/cm² irradiation caused higher cell damage than the optimum irradiation level which initiates the cell damage with no significant cell loss.

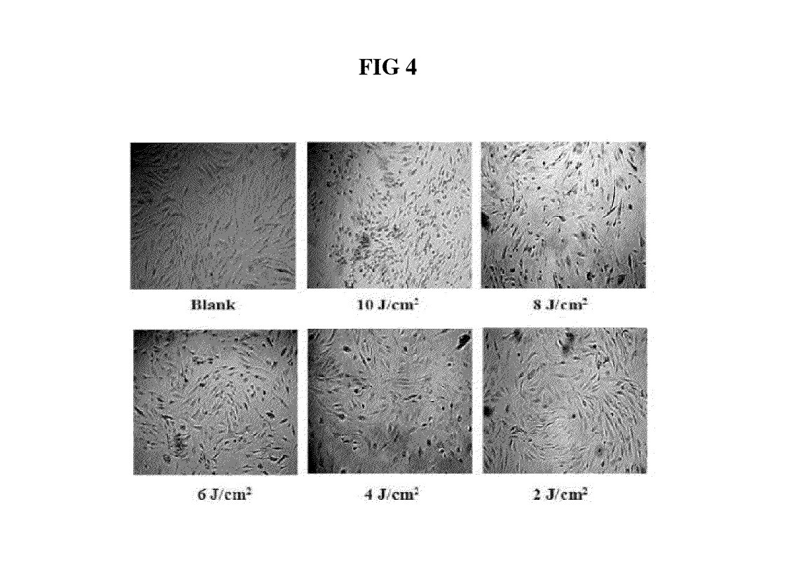

As illustrated in FIG. 4, changes in dermal fibroblasts size and shape were observed by phase-contrast microscopy. Cultured dermal fibroblasts displayed a typical morphology for fibroblast cells, with an elongate shape. The dermal fibroblasts exhibited a normal monolayer culture type, which had the characteristics of contact inhibition, when cells started to become fully confluent. In morphological assay, morphological changes in dermal fibroblasts induced by UV-A treatment were examined with an inverted phase-contrast microscope at different dosage of UV-A irradiation. Incubation time was chosen as 24 h from previous results and the morphological observation assay strengthen the results of MTT assay. Cells were subjected to continuous UV-A irradiation for 24 h and the morphological changes were reported. In accordance with the previous results, smaller dosages than 6 J/cm² failed to induce the desired morphological changes and 8 and 10 J/cm² damaged the morphology more than desired. Therefore, the 6 J/cm² choose for the optimum UV-A irradiation was again showed to be the reliable process followed by the 24 h incubation.

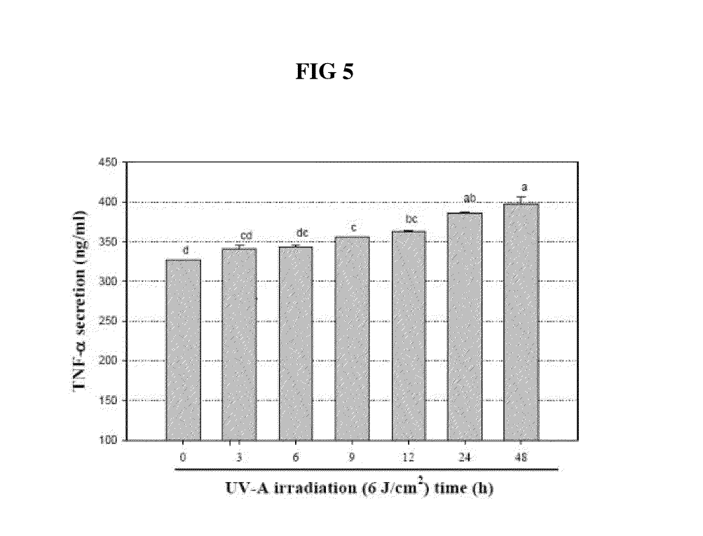

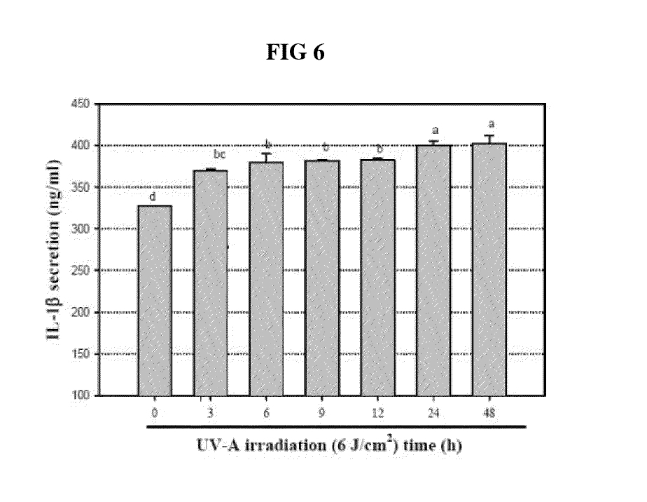

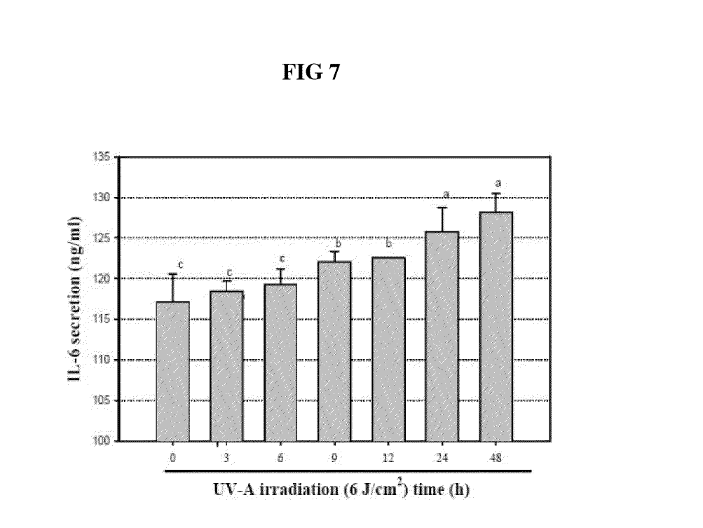

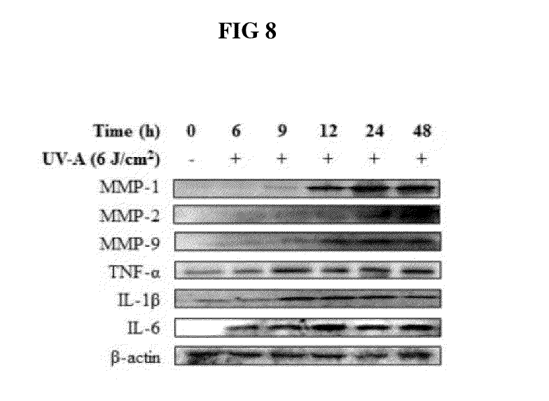

Activation of cells by UV-A irradiation is followed by increased secretion of cytokines such as TNF-α, IL-1β and IL-6. Therefore, the promote the 24 h as the optimum incubation time for UV-A irradiated activation of cells, secretion of above mentioned cytokines were examined by ELISA. Cells were subjected to continuous UV-A irradiation at the dosage of 6 J/cm² for various incubation time. Secretion of TNF-α, IL-1β and IL-6 did not show a significant change before 24 h incubation (see FIGS. 5 to 7). Twenty four hours of incubation initiated the activation of cells in respect to cytokine secretion. Hence, the optimum time was again chosen as the optimum incubation time. In addition, to determine the best time point for detecting UV-A mediated gene expression, the levels of MMPs expressions were compared using western blot analysis in a time-dependent manner because UV-A irradiation was also reported to elevate the MMP expression. As illustrated in FIG. 8, the expression level of MMP-1 was gradually increased according to time of UV-A irradiation. Therefore, activation of cells by UV-A irradiation for 24 h might be the optimal time for intracellular MMP detection when taken into consideration along the results from previous assays.

As a consequence, according to all results conducted to determine the optimum incubation time and UV-A irradiation dosage, 6 J/cm² of UV-A irradiation followed by 24 h incubation was observed as the optimum conditions for reliable assays in order to determine the protection of cells from UV-A irradiated cell activation. All assays results were supported this choice, hence further experiments for testing compounds for their protection was carried out with 24 h incubation time period and continuous exposure of 6 J/cm² UV-A irradiation unless stated otherwise.

### Experimental Example 2

### Chromenes Having Protective Effect on UV-A Induced Dermal Fibroblasts Viability

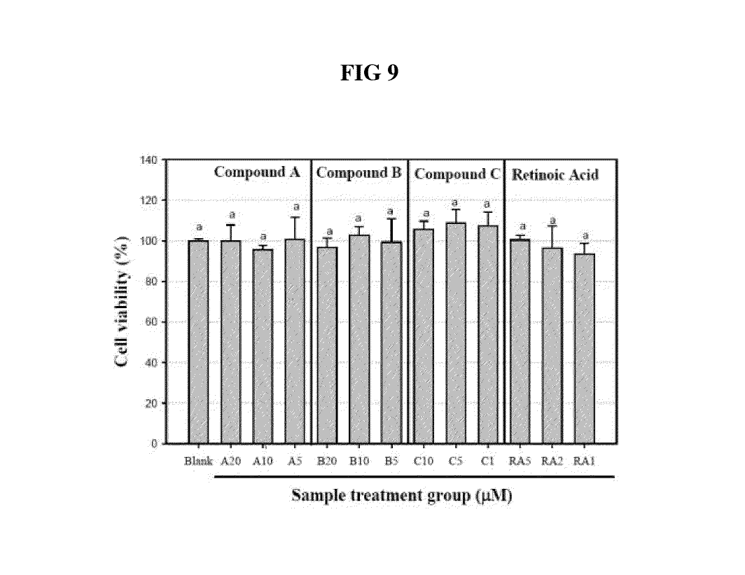

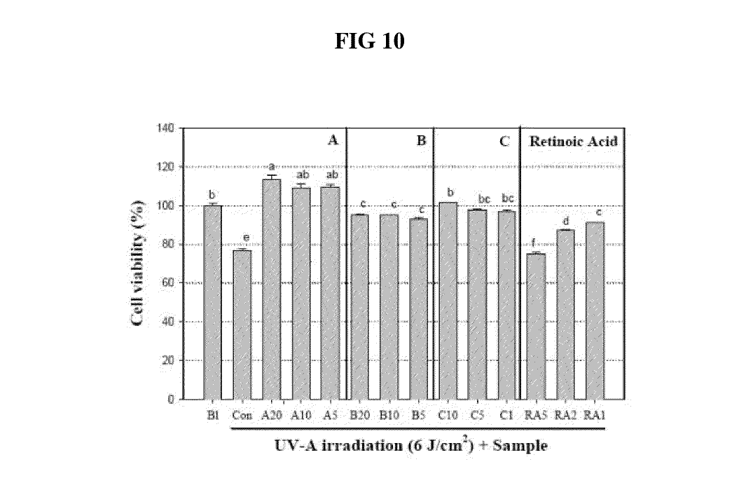

None of the compounds exhibited any significant cytotoxicity as illustrated in FIG. 9. Results obtained from MTT assay revealed that compounds isolated from Sargassum horneri are safe compounds for in vitro experiments. Furthermore, we checked the protective effect of compounds on UV-A induced cell damage. The result has shown to FIG. 10, treatment with compounds were significantly protected cell damage in a dose-dependent manner.

### Experimental Example 3

### Phototoxic Effect of Chromenes

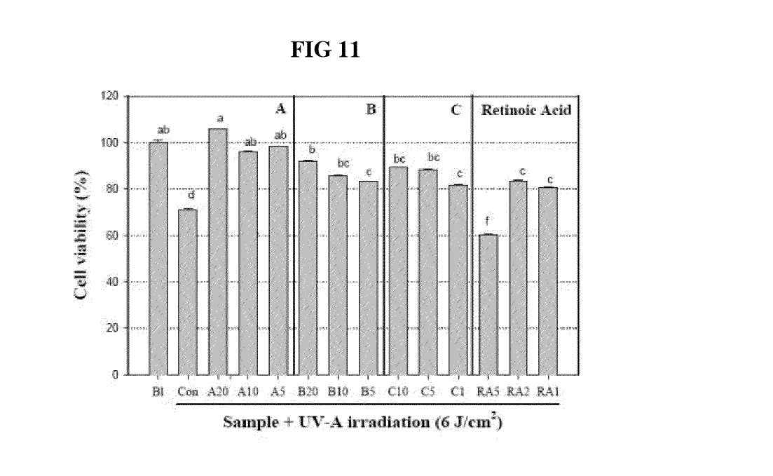

In order to determine whether compounds have phototoxic effect, the cell viability of dermal fibroblasts was evaluated using MTT assay. Cells were co cultured with compounds and retinoic acid during UV-A irradiation. As shown in FIG. 11, none of the compounds exhibited any significant phototoxicity whereas retinoic acid showed a little phototoxic effect at the concentration of 5 μM. Therefore, the concentration of 1 μM retinoic acid was selected as a positive control for further experiment.

### Experimental Example 4

### Chromenes Inhibited Intracellular ROS Generation Caused by UV-A Irradiation

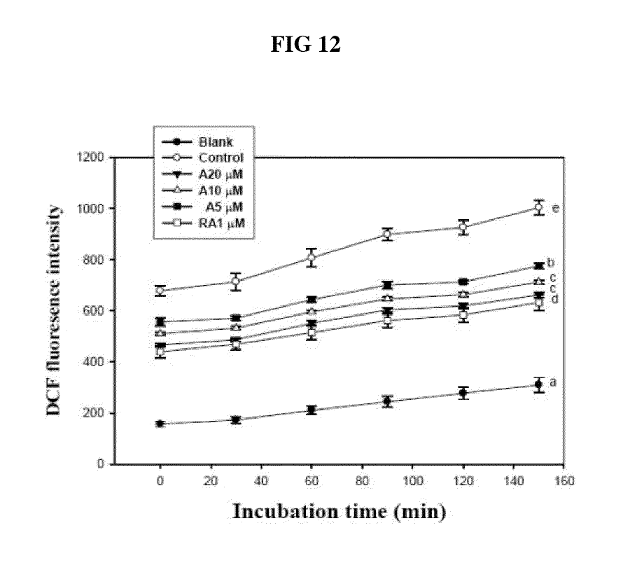

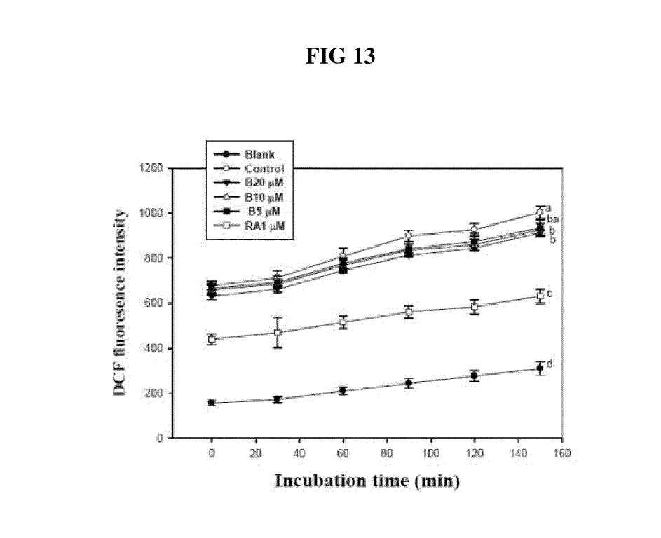

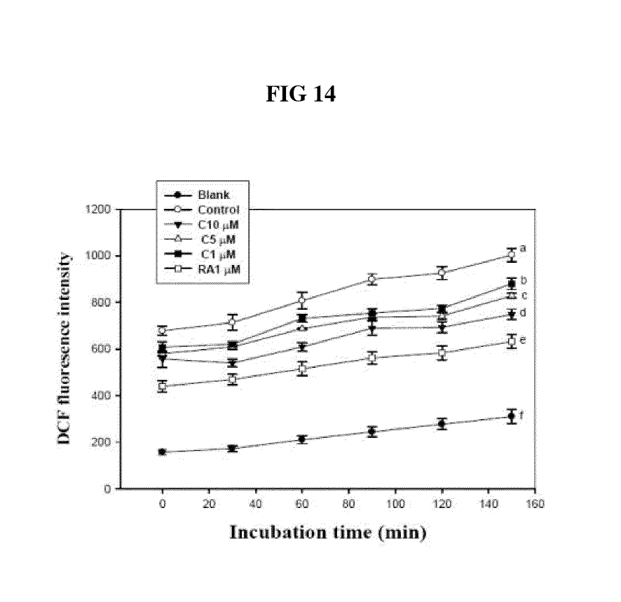

Intracellular radicals scavenging effect of chromenes was determined in time and concentration-dependent manners using fluorescence sensitive dye, DCFH-DA (FIGS. 12 to 14). This method has generally been used to monitor intracellular ROS production. The cell system was labeled with DCFH-DA and followed by UV-A irradiation. The progressive increments in DCF fluorescence intensity due to intracellular ROS generation were observed with the incubation time of up to 150 min. Treatments with the chromenes led to a significant reduction of DCF fluorescence intensity, resulting in increased scavenging activity against intracellular ROS formation in a concentration-dependent manner (p<0.05). Among the chromenes, compound C was the most effective than other compounds in the suppression of UV-A induced ROS generation compared to control group (UV-A alone treated group). Accordingly, in the presence of chromenes a remarkable reduction of ROS generated due to UV-A irradiation was observed compared to UV-A only treated control group.

### Experimental Example 5

### Inhibition Effect of Cell Membrane Protein Oxidation

Proteins are known to be important targets for oxidative modifications. Oxygen radicals and other activated oxygen species cause modifications of the amino acids of proteins that frequently result in functional changes of structural or enzymatic proteins. Protein carbonyls may be formed either by oxidative cleavage of proteins or by direct oxidation of lysine, arginine, proline, and threonine residues. In addition, carbonyl groups may be introduced into proteins by reactions with aldehydes (4-hydroxy-2-nonenal, malondialdehyde) produced during lipid peroxidation or with reactive carbonyl derivatives generated as a consequence of the reaction of reducing sugars or their oxidation products with lysine residues of proteins. Carbonyl residues can be readily detected by reaction with 2,4-dinitrophenylhydrazine (DNPH) generating dinitrophenylhydrazones. The detection of carbonyl groups in skin has therefore been used as a marker of reactive-oxygen-mediated protein oxidation. UV-A exposure hypothesized that deplete antioxidant defense and induce protein oxidation in dermal fibroblasts.

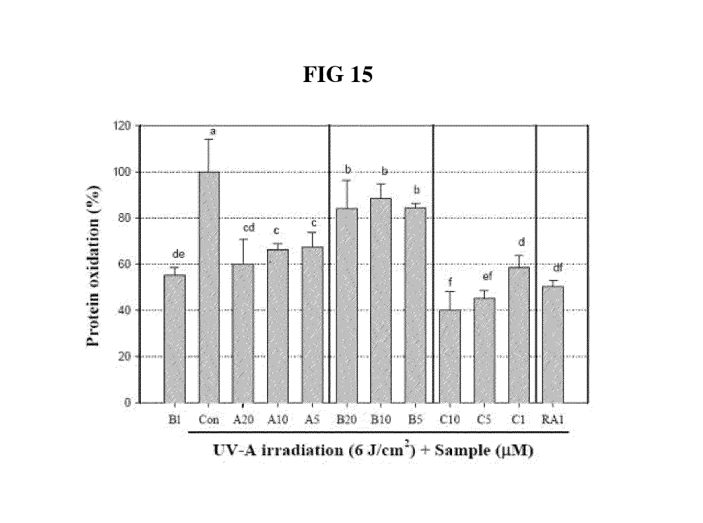

The result was shown in FIG. 15, a clear reduction in carbonyl groups formation was observed following the treatment with chromenes to the reaction mixture containing dermal fibroblasts membrane. Their high potency inhibition against protein oxidation exhibited in a concentration-dependent pattern. Among the chromenes, compound C was the most effective in the prevention of membrane protein oxidation induced by UV-A irradiation.

### Experimental Example 6

### Chromenes Having Protective Effect on Cell Membrane Lipid Peroxidation Caused by UV-A Irradiation

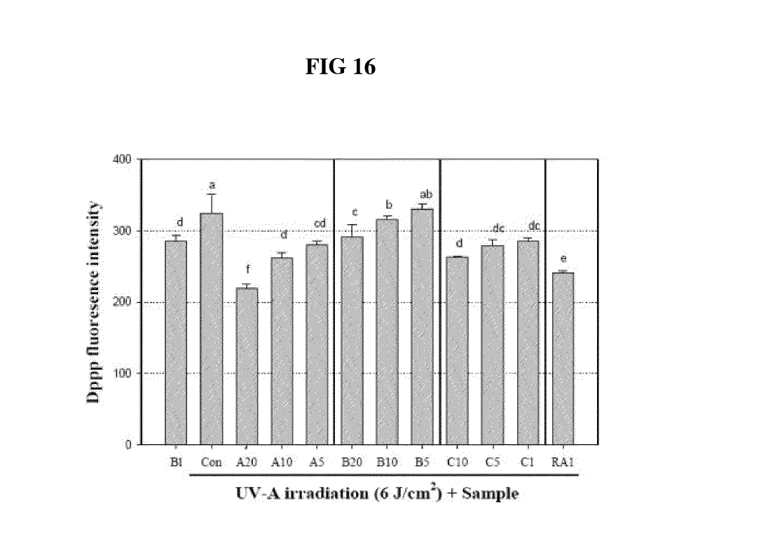

The levels of lipid hydroperoxides in the presence and absence of chromenes were examined by using specific fluorescence probe, DPPP (FIG. 16). DPPP has been used as a sensitive method for measuring lipid hydroperoxides of cell membranes, since it successfully incorporates into membranes and oxidizes hydroperoxides to emit DPPP-oxide fluorescence. When the DPPP-labeled fibroblasts were treated with UV-A irradiation, the fluorescent intensity derived from DPPP oxide steadily increased due to UV-A mediated membrane lipid peroxidation. Treatment with chromenes led to a concentration-dependent reduction in the fluorescent intensity. The reduction in fluorescent intensity was compared to the blank (UV-A non-stimulated), which confirming that chromenes could exert a substantial effect against oxidation of membrane lipids. Among the chromenes, Compound A and C have similar effect on the inhibition of membrane lipid peroxidation caused by UV-A irradiation.

### Experimental Example 7

### Chromenes Enhanced GSH Level in UV-A Irradiated Dermal Fibroblasts

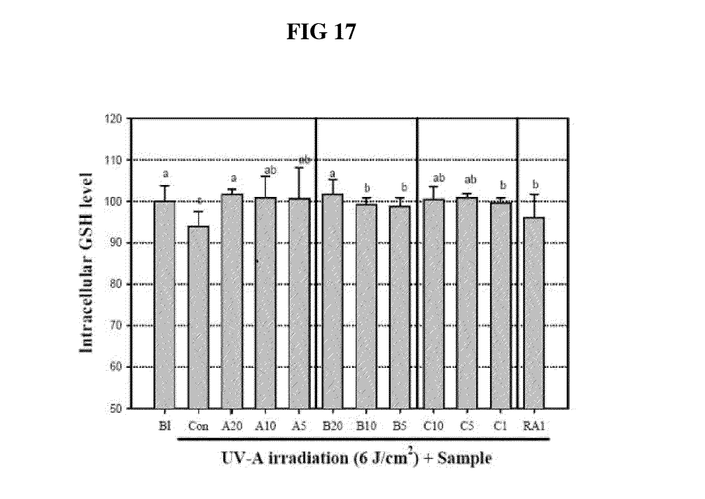

GSH is the most abundant low molecular weight thiol inside mammalian cells, and changes in glutathione level directly reflect intracellular redox alterations. To investigate the relationship between the increased ROS and the level of antioxidant materials in cells, the intracellular GSH level was measured. Dermal fibroblasts were labeled with thiol reactive fluorescent dye, mBBr for the determination of GSH level in the presence or absence of compounds after UV-A irradiation and results are presented as fluorescence intensity (FIG. 17). Intracellular GSH levels were significantly increased in the presence of three compounds, compared with those in the presence of UV-A irradiation as shown in FIG. 17. The results of this study demonstrated that the level of GSH was much higher in chromenes treated cells than retinoic acid treatment. Moreover, the level of GSH was shown to be increased by treatment of chromenes in a dose dependant manner reaching the blank group after 24 h. The level of GSH was also increased slightly with the treatment of retinoic acid. Interestingly, at the 1 μM concentration of retinoic acid did not exhibit a satisfactory increment in GSH level other than chromenes treatment.

### Experimental Example 8

### Chromenes Inhibit Production of Inflammatory Cytokines by UV-A

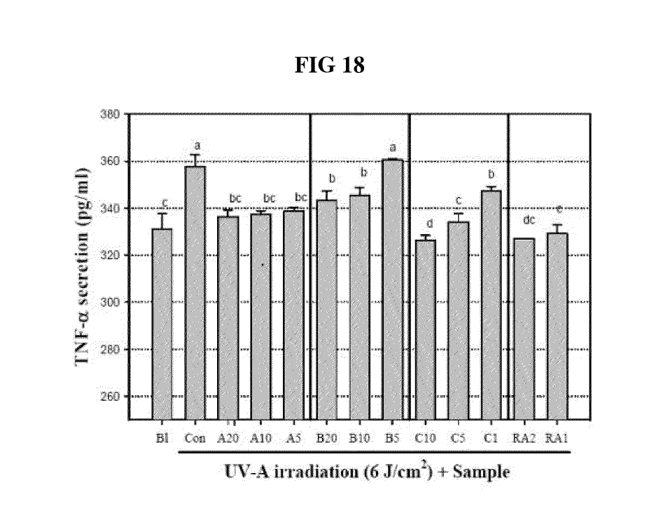

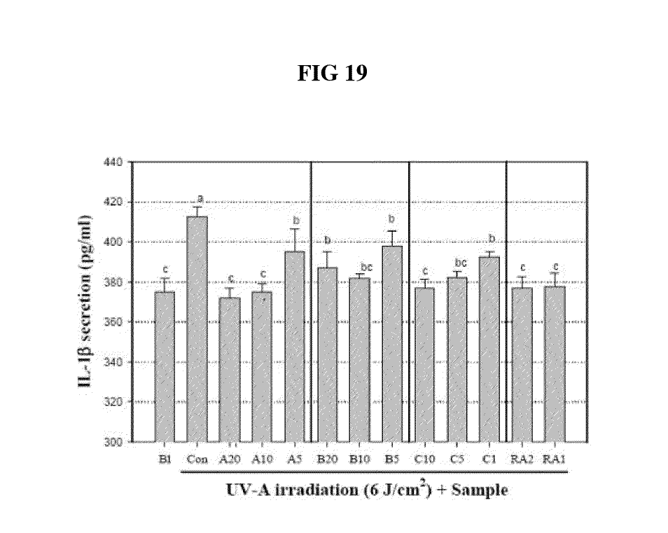

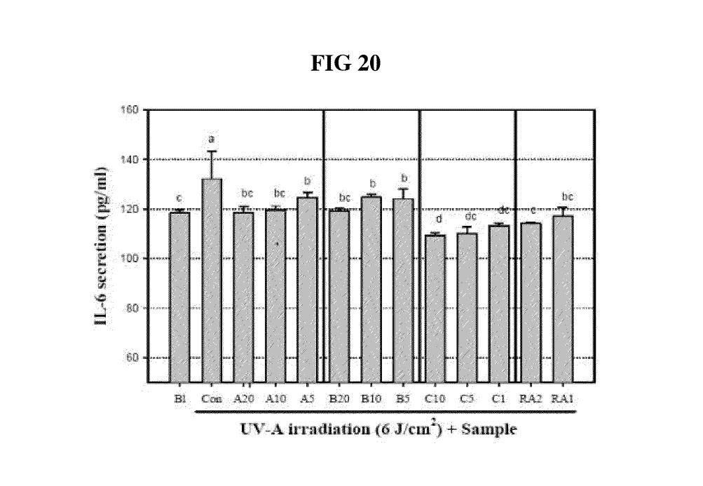

To investigate effect of chromenes on the cytokine production in UV-A irradiated dermal fibroblasts, inflammatory cytokine production was measured using ELISA assay (FIGS. 18 to 20). UV-A activation of dermal fibroblasts induced the secretion of pro-inflammatory cytokines, including TNF-α, IL-1β and IL-6. However, treatment with chromenes decreased secretion levels in the cell cultured media of UV-A-activated fibroblasts, compare to groups of UV-A irradiation and retinoic acid treatment. Compounds A, B and C were treated in various concentrations from 5 to 20 μM in case of A and B and 1 to 10 μM in case of C according to their activity which was screened earlier. All three compounds exhibited a reducing effect on the elevated cytokine secretion compared to control group which was irradiated only without any treatment. Among three compounds, C was the most active one in respect to positive control retinoic acid. In case of all tested cytokines, TNF-α, IL-1β and IL-6, C was observed to show a similar effect with that of retinoic acid. However, C showed the same effect at the concentration of 10 μM, while retinoic acid reduced the cytokine secretion in same level at a concentration of 2 μM. Compound A and B slightly reduced the cytokine secretion in a barely dose-dependent manner, compound C inhibited the secretion of TNF-α and IL-1β in significant levels with a clear dose-dependency. On the other hand, it showed a lesser dose-dependency in case of IL-6, yet a quite similar inhibition with the positive control retinoic acid. Moreover, compound C decreased the secretion of TNF-α and IL-6 which are lower than the untreated and unirradiated blank groups at the concentration of 10 μM. Additionally, 1 μM of compound C treatment lowered the elevated IL-6 levels to 115 ρg/ml from 135 ρg/ml while the retinoic acid could reduce it to 119 pg/ml at the same concentration. These results suggested that the presence of chromenes effectively attenuated the secretion of inflammatory cytokines by UV-A irradiation in dermal fibroblasts.

### Experimental Example 9

### Chromenes Inhibited Inflammatory Response Gene and Protein Expression

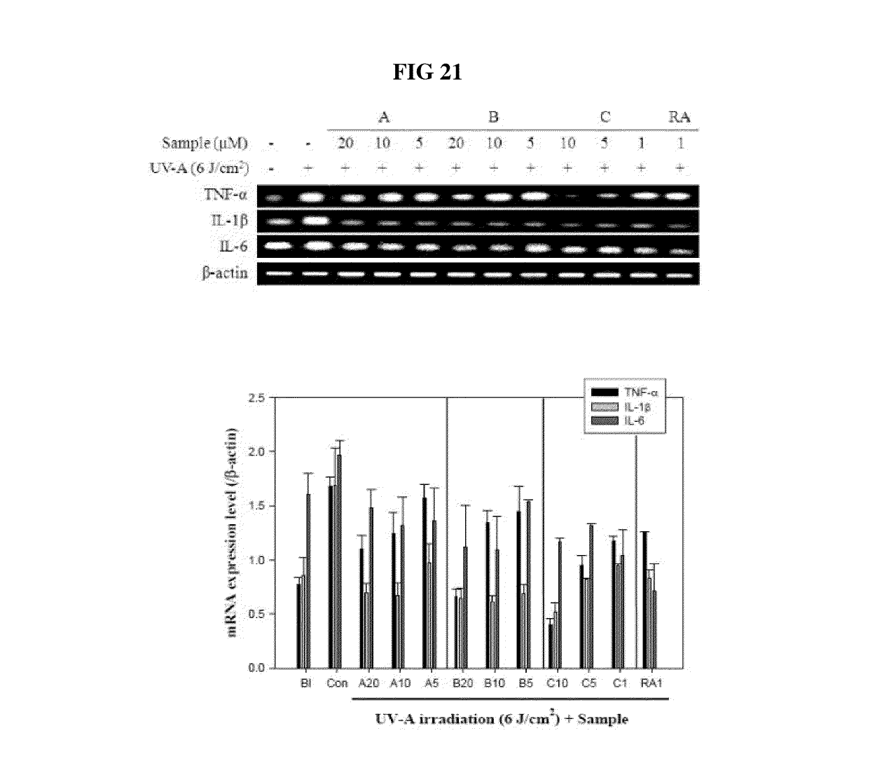

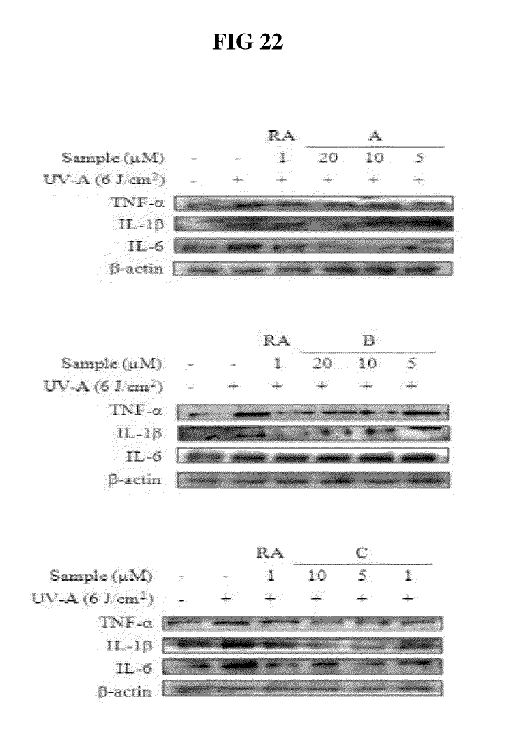

We confirmed whether chromenes can affect the inflammatory response cytokine mRNA transcription and protein expression in UV-A irradiated dermal fibroblasts, inflammatory gene and protein levels were determined by RT-PCR and Western blot analysis, respectively (FIGS. 21 and 22). The mRNA transcription and protein levels of TNF-α, IL-1β and IL-6 were reduced by chromenes treatment, which is consistent with the results obtained from cytokine secretions. In accordance with the previous ELISA assay results for cytokine secretion, compound C showed the highest efficiency to reduce the expression of inflammatory response genes and proteins. In terms of inhibition the expression of TNF-α, IL-1β and IL-6 all three compounds were able to show some activity. However, compound C clearly inhibited the TNF-α expression while compound A and B failed to show significant activity. On the other hand, compound A and B showed similar effect with compound C on inhibiting IL-1β and IL-6 expression. Western blot analysis showed parallel results with the RT-PCR assay. Compound C was again observed as the most active one to inhibit the production of TNF-α, IL-1β and IL-6 proteins compared to compound A and B, which also showed an inhibitory effect however not as efficient as the compound C. In addition, in all cases, gene and protein expression, efficiency of compound C was similar to that of retinoic acid at the highest concentration treatment, which is 10 μM for compound C and 1 μM for retinoic acid.

### Experimental Example 10

### Chromenes Inhibited UV-A Induced Hyarulonan Secretion

Hyaluronan (also called hyaluronic acid or hyaluronate) is also a major component of the extracellular matrix, where it is involved in tissue repair. Hyaluronan degradation can be start when skin is exposed to excessive UV light, it becomes inflamed (sunburn) and the cells in the dermis reduced the hyaluronan production by increase the rate of its degradation.

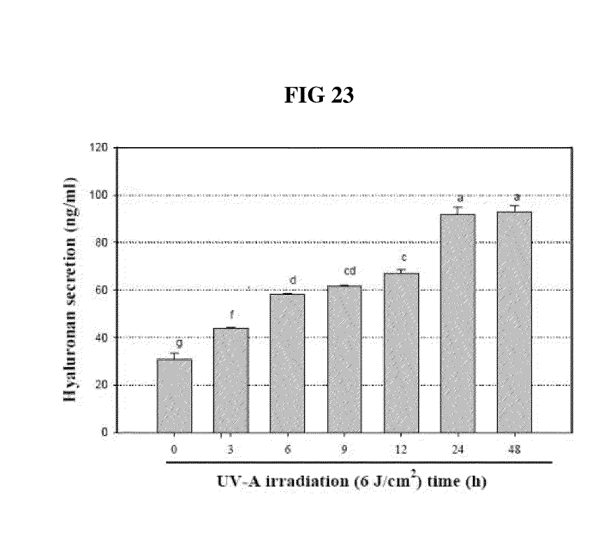

As shown in FIG. 23, hyaluronan secretions were increased continuously time-dependent manner. We obtained that hyaluronan secretions were significantly increased especially in time point of 24 h. Therefore, 24 h was decided as a suitable time point to detect the hyaluronan contents. Then, we evaluated effect of chromenes on hyaluronan secretion by UV-A irradiation. The result addressed in FIG. 23, hyaluronan secretion was increased by UV-A irradiation whereas this level was reduced by chromenes treatment. Hyaluronan secretion level was successfully inhibited by treatment with retinoic acid at the concentration of 1 μM. Interestingly, this inhibition level was similar with effect of compound C followed by compound A and compound B. These results indicated that hyaluronan secretion caused by UV-A irradiation was negatively regulated by chromenes treatment. Therefore, treatment of chromenes might be an effective strategy for the protection of UV-A induced hyaluronan degradation.

### Experimental Example 11

### Chromenes Inhibited mRNA Expression of Hyaluronan Syntheases Induced by UV-A Irradiation

Hyaluronan is synthesized by a class of integral membrane proteins called hyaluronan synthases (HASs), HAS1, HAS2 and HAS3. It has been reported that hyaluronan synthesis inhibited by 4-methylumbelliferone (hymecromone, heparvit), a 7-hydroxy-4-methyl coumarin derivative (Kakizaki et al., 2004). Without inhibiting other glycosaminoglycans, inhibition of hyaluronan synthases may suggest useful in prevention of skin damage caused by UV-A irradiation (Kakizaki et al., 2008).

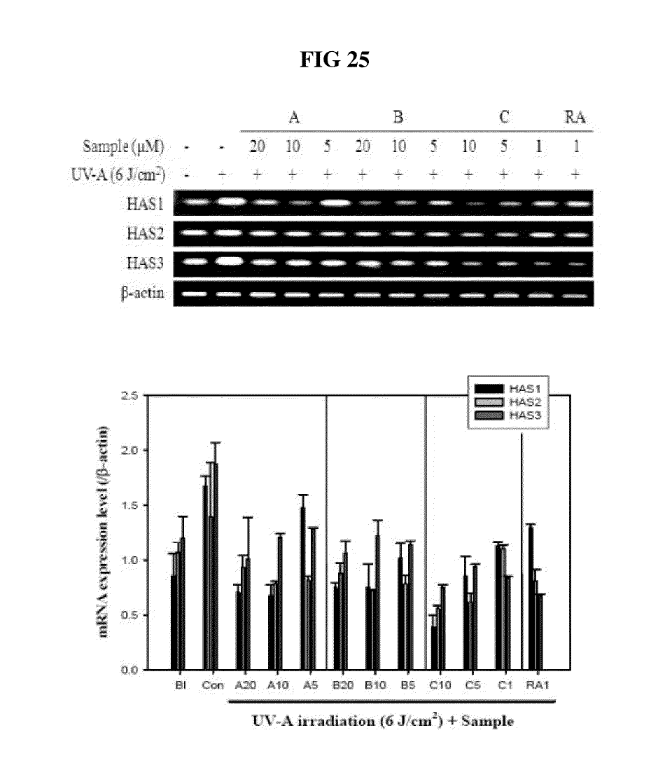

As shown in FIG. 25, the mRNA expression levels of hyaluronan synthases such as HAS1, HAS2 and HAS3 were significantly increased 6 J/cm² of UV-A irradiation after 24 h. These expressions were proportional to the hyaluronan contents obtained from ELISA experiment. The results revealed that mRNA expression levels of hyaluronan synthases were down-regulated after treatment with chromenes in UV-A irradiated dermal fibroblasts. Effect of compound C on the inhibition of hyaluronan syntheases was similar with retinoic acid treatment followed by compound A and compound B. Therefore, chromenes have inhibitory effect on inflammatory cell damage caused by UV-A irradiation via regulation of hyaluronan synthases.

### Experimental Example 12

### Chromenes Inhibited Elastase

Fibroblast elastase belongs to the metalloproteinase family. Phosphoramidon is known as a typical inhibitor of metalloproteinase, but it has poor permeability through the skin because of the presence of the hydrophilic rhamnose residue. Loss of skin elastin caused by UV-A irradiation results in wrinkle formation. Therefore, the chromenes were investigated for their anti-elastase activities using N-Succ-(Ala)3-p-nitroanilide as the substrate for elastase and examined for their inhibition activities by monitoring the absorbance at 410 nm due to the release of p-nitroaniline. The results are summarized in the following Table 6. Among three compounds, compound C showed the most strong elastase inhibition activity (IC50 6.78 μM), which is almost two times more active than compound A and compound B.

Retinoic acid showed the moderate activities at the IC50 of 1.04 μM.

TABLE 6
Anti-elastase activity

| | Elastase inhibition IC₅₀ (μM ± SD) |
|---|---|
| Compound A | 11.28 ± 0.13 |
| Compound B | 13.77 ± 0.13 |
| Compound C | 6.78 ± 0.12 |
| Retinoic acid | 1.04 ± 0.10 |

### Experimental Example 13

### Inhibitory Effect of Chromenes on UVA-Induced Collagen Degrading MMP Secretions

Collagens are known to as main component of the skin and degraded by collagenase MMPs. Among MMPs, MMP-1, MMP-8 and MMP-13 are family of collagenases which are capable of degrading triple-helical fibrillar collagens. Loss of skin collagen resulted in wrinkle formation. Therefore, inhibitory effect of chromenes on collagenase MMP secretions from the UV-A irradiated dermal fibroblasts was determined using ELISA assay.

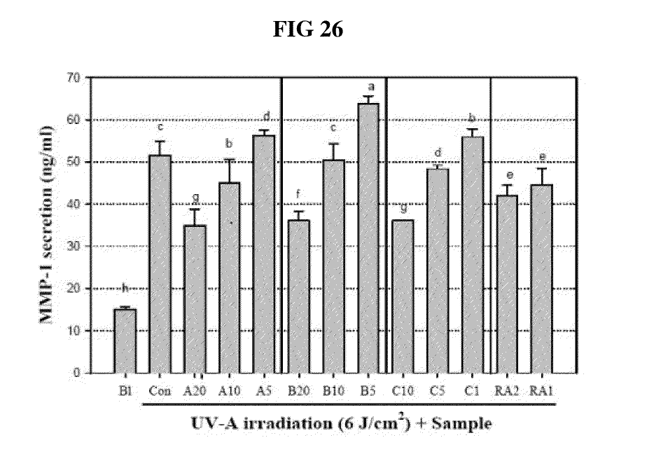

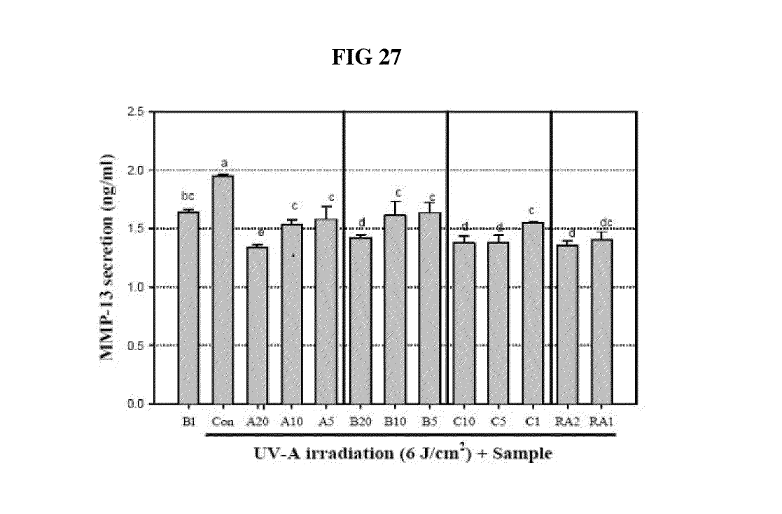

As shown in FIGS. 26 and 27, MMP-1 and MMP-13 secretions were reduced in the presence of chromenes in a dose-dependent manner. Among three compounds, C was the most active one in respect to positive control retinoic acid. In case of MMP-1 was observed to show a similar effect with that of retinoic acid. However, C showed the same effect at the concentration of 10 μM while retinoic acid reduced the MMP-1 secretion in same level at 1 μM. Compound A and B significantly reduced the MMP-1 and MMP-13 secretions in a dose-dependent manner, compound C inhibited the secretion of MMP-1 and MMP-13 with the highest significance compare with other two compounds. Moreover, compound C decreased the secretion of MMP-1 and MMP-13 to levels which are lower than the untreated and unirradiated blank groups at the concentration of 10 μM. These results confirmed that chromenes exerted adequate protective effect on collagen degradation by UV-A irradiation.

### Experimental Example 14

### Chromenes Enhanced the Procollagen Level

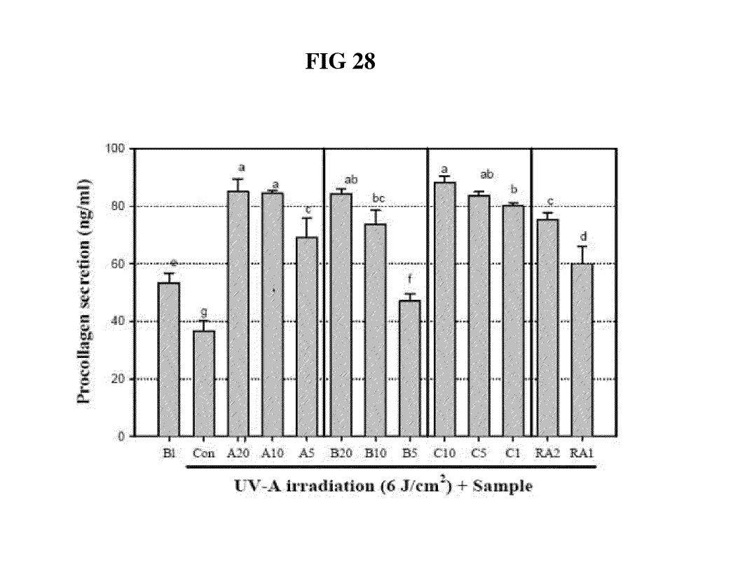

Alterations of collagen have been thought to be a cause of the clinical skin changes of photoaging. The dermis contains predominantly type I collagen (85-90%) and the precursor molecules of collagen are called procollagen, which is synthesized in the form of polypeptide chains of type I collagen in dermal fibroblasts. UV irradiation induces the activity and synthesis of MMP in human skin, which mediate collagen destruction that results in much connective tissue damage. Therefore, we also studied the effects of chromenes on the level of type I procollagen in UVA-irradiated cultured dermal fibroblasts. The procollagen level was determined in the culture medium by ELISA assay. Type I procollagen secretion level was increased by 81, 86 and 91 ng/ml in the presence of compound C at the concentration of 1 μM, 5 μM and 10 μM, respectively (FIG. 28). Retinoic acid has been shown to have anti-aging properties, was used as a control drug. Treatment of 1 μM of retinoic acid decreased MMP-1 expression and increased the level of type I procollagen up to 60 ng/ml. In comparison of compound C and retinoic acid, compound C was more effective in procollagen secretion than retinoic acid at the same concentration of 1 μM. Thus, chromenes have protective effects on collagen degradation by enhancement of collagen synthesis in photo-damaged dermal fibroblasts.

### Experimental Example 15

### Chromenes Suppressed UV-A-Induced Collagen Degrading MMP Expressions

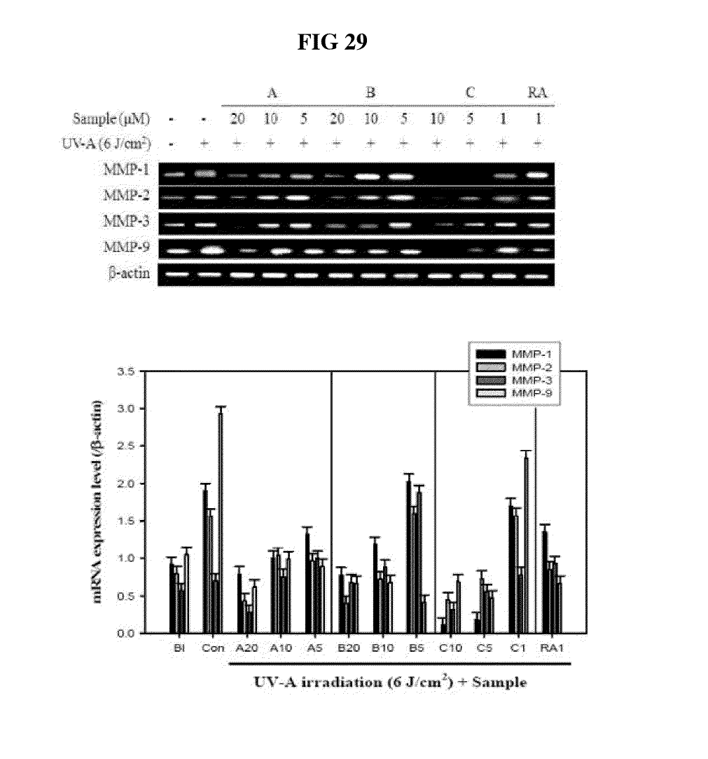

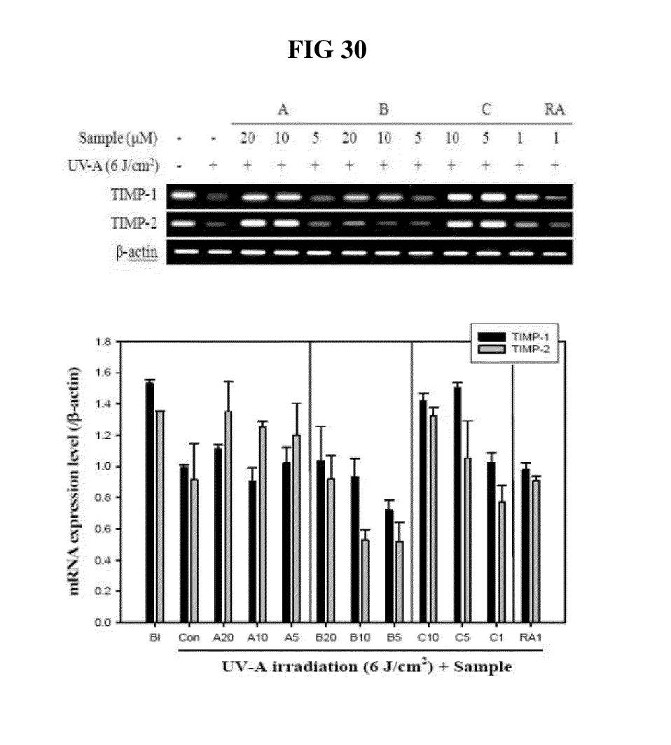

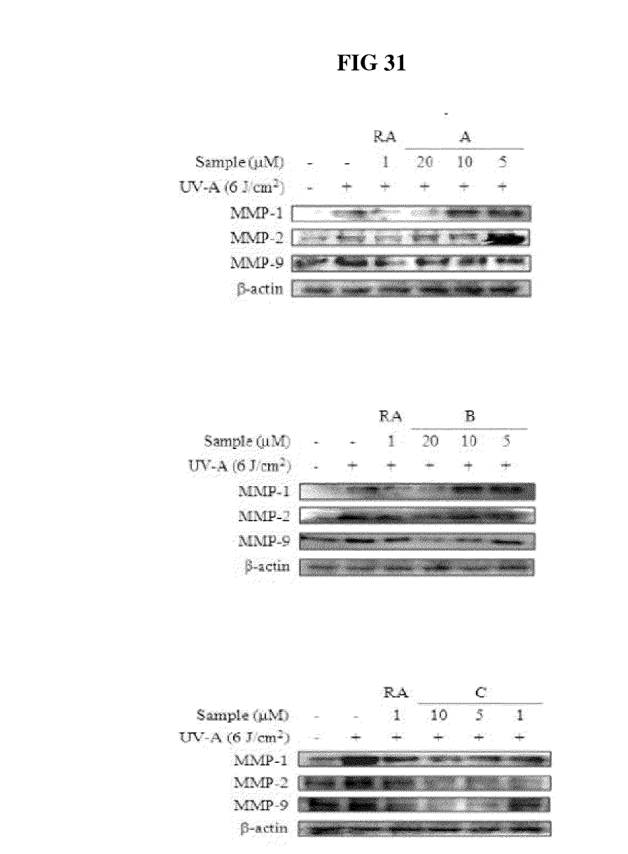

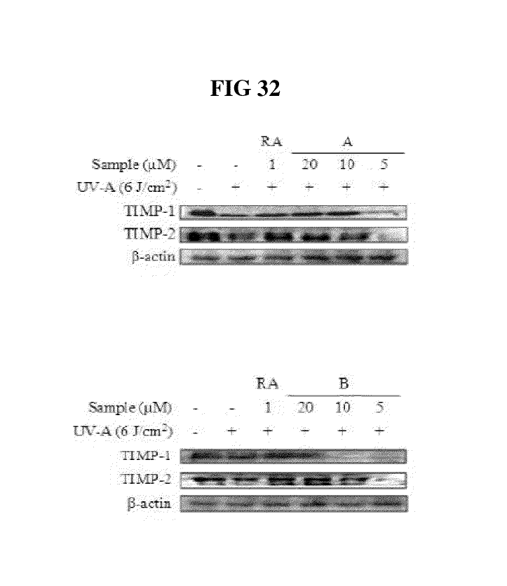

Effects of chromenes on collagenase MMPs expression in UV-A irradiated dermal fibroblasts were determined using RT-PCR and Western blotting analysis (FIGS. 29 to 32). The collagenase MMP gene and protein expression levels of MMP-1, MMP-2, MMP-3 and MMP-9 were suppressed by treatment with chromenes in a concentration dependent manner. Moreover, these collagenase levels were regulated by TIMPs genes. The levels of TIMP-1 and TIMP-2 gene were reduced by UV-A irradiation whereas the levels of TIMPs genes were enhanced by treatment with chromenes. All three compounds exhibited a reducing effect on the elevated MMPs expression both in the gene and protein levels compared to control groups which was irradiated only without any treatment. Among three compounds, C was the most active one in respect to positive control retinoic acid. In case of effect of compound C on all tested MMPs expression, MMP-1, MMP-2, MMP-3 and MMP-9 were observed to have a similar effect to that of retinoic acid. According to these results, chromenes were exhibited protective effect on UV-A induced collagen degradation via down-regulation of MMPs expression.

### Experimental Example 16

### Chromenes Enhanced Elastic Fiber Expression in UV-A Irradiated Fibroblasts

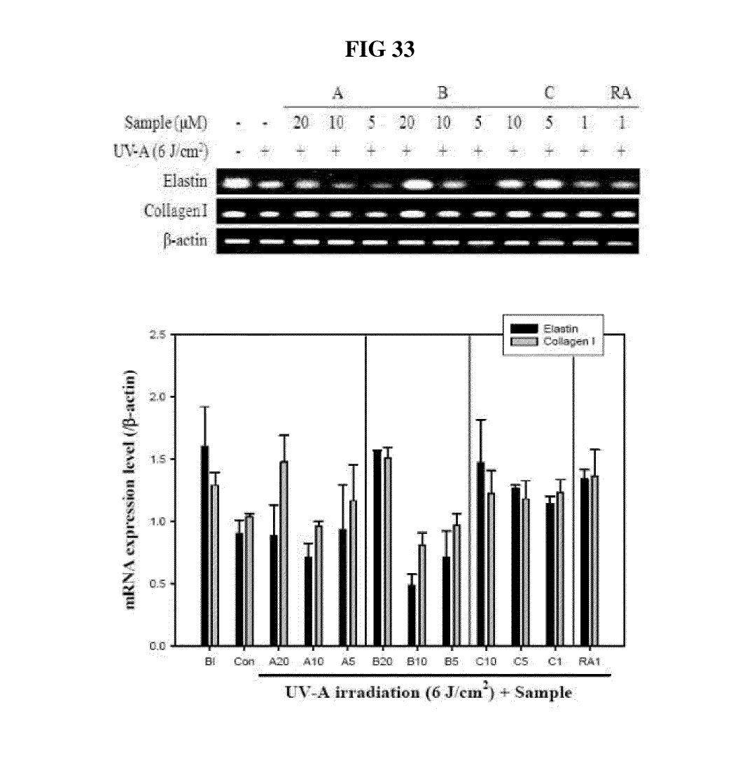

In order to evaluate whether chromenes can affect elastic fiber expression in UV-A irradiated dermal fibroblasts, synthetic markers of elastic fiber were examined by RT-PCR and Western blot analysis, respectively (FIG. 33). Collagen type I, is the most abundant component of skin, type I collagen is significantly degraded by UV exposure thereby resulted in skin wrinkling. Treatment with chromenes increased collagen type I levels in a dose-dependent manner. Furthermore, regulation of procollagen was decreased in UV-A alone exposed fibroblasts. However, these decreased cellular levels of procollagen due to UV-A exposure were increased in the presence of chromenes.

Elastin is an important protein in connective tissue that allows many tissues in the body to resume their shape after stretching or contracting. Additionally, elastin helps skin to return to its original position when it is poked or pinched. It has been reported that the level of elastin was significantly reduced during UV irradiation. Thus, loss of skin elastin resulted in wrinkle formation caused by UV. Present study revealed that expression level of elastin was enhanced followed by chromenes treatment. Moreover, compound C showed the highest activity compare to other compounds in most case of elastic fiber expression such as collagen type I and elastin. These results indicated that chromenes might prevent loss of skin elasticity by increment of the elastin and collagen expression due to reducing the UV-A induced elastic fiber degradation.

### Experimental Example 17

### Chromenes Enhanced Decomposition of Extracellular Matrix Components Caused by UV-A

In order to determine the protective effect of compound A, B and C on UV-A irradiation-induced decomposition of extracellular matrix components, an immunofluorescent labeling assay was carried out. It has been reported that the exposure to UV-A causes degradation of collagen and fibronectin in the extracellular matrix. This decomposition of extra cellular components is followed by increased levels of hyaluronan synthase 1 in UV-A irradiated. Under these circumstances the effects of compound A, B and C on the fluorescent labeled (green) collagen, fibronectin and hyaluronan synthase 1 were tested along UV-A irradiation and compared with blank and control groups. Results were evaluated through the images of the labeling assay under a fluorescent microscope with the previously mentioned excitation and emission values. For all labeling assays retinoic acid was used as a positive control in addition to Hoechst stain (blue) which is an indicator of viable nucleus.

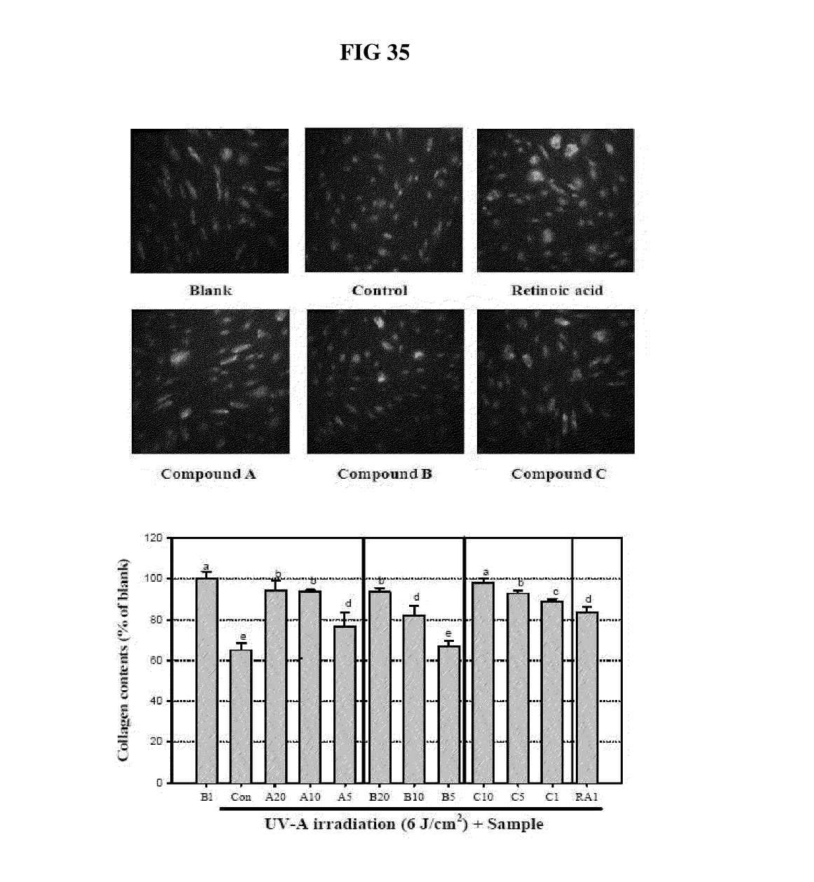

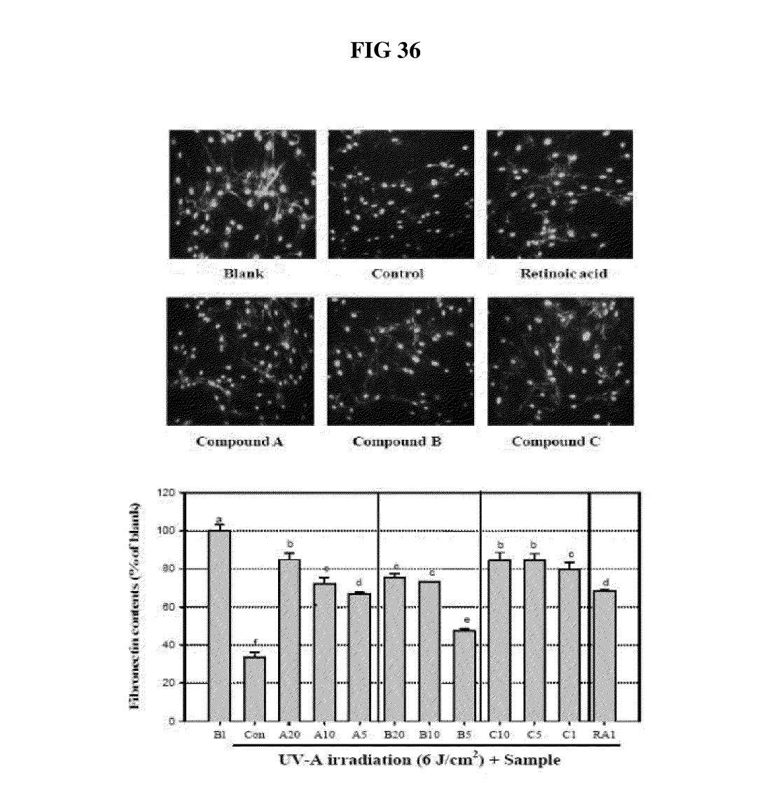

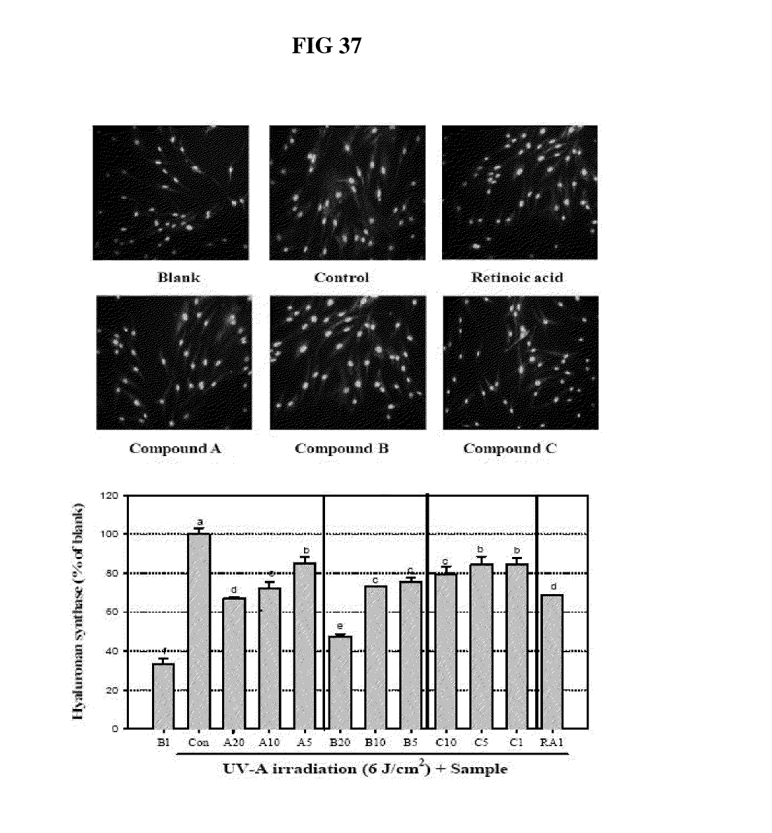

All three compounds were observed to protect the collagen degradation to a level under UV-A irradiation (FIG. 35). Among three treatments, compound C showed the highest protection which is at the same level as retinoic acid. The efficiency of compound is followed by compounds A and B, respectively in respect to amount of undegraded collagen. In accordance with collagen labeling assay, compound C was shown to protect the decomposition of fibronectin as effective as the retinoic acid (FIG. 36). Besides, compounds A and B also showed a protection activity, however not as significant as compound C. It was observed that UV-A irradiation increased the amount of hyaluronan synthase 1 (FIG. 37). Treatment with compounds A, B and C reduced the increased hyaluronan synthase 1 amount according to immunofluorescent labeling. Parallel to all results obtained, compound C caused the highest reduction of hyaluronan synthase 1 amount which is again at the same level with a positive control, retinoic acid. In summary, all compounds showed a protective effect against UV-A irradiated decomposition of the extracellular matrix by protecting the collagen and fibronection degradation as well as reducing the hyaluronan synthase 1 level. Among three treated compounds, compound C was observed as the most effective agent to protect extracellular matrix from UV-A irradiation.

### Experimental Example 18

### Chromenes Inhibited UV-A Induced AP-1 Signal Pathway

The present study was evaluated AP-1 activation to understand the action of chromenes on anti-aging profile. AP-1 is composed of jun and fos family proteins. It has been known to inhibit transcription of procollagen gene by induction of MMP transcription, which is responsible for the degradation of collagen. The results demonstrated that UV-A irradiation of the human dermal fibroblasts induced the expression of several AP-1 family members including c-Fos and c-Jun.

As shown in FIG. 57, chromenes contributed to negative regulation of UV-A induced AP-1 activation as well as UV-A induced the regulation of c-Jun and c-Fos proteins induced by UV-A irradiation. Especially, compound C has a similar effect with retinoic acid on UV-A induced AP-1 activation. Therefore, the result demonstrated that chromenes played important roles in UV-A mediated AP-1 activation as well as down-regulation of c-jun and c-fos protein expression in dermal fibroblasts.

### Experimental Example 19

### Chromenes Enhanced Down-Regulation of TGF-β/Smad Signaling by UV-A Irradiation

UV-A mediated down-regulation of collagen synthesis also occurs via signaling pathways involving TGF-β. As a major profibrotic cytokine, TGF-β regulates various cellular functions involving differentiation, proliferation and enhancement of synthesis of extracellular matrix such as collagen and elastin.

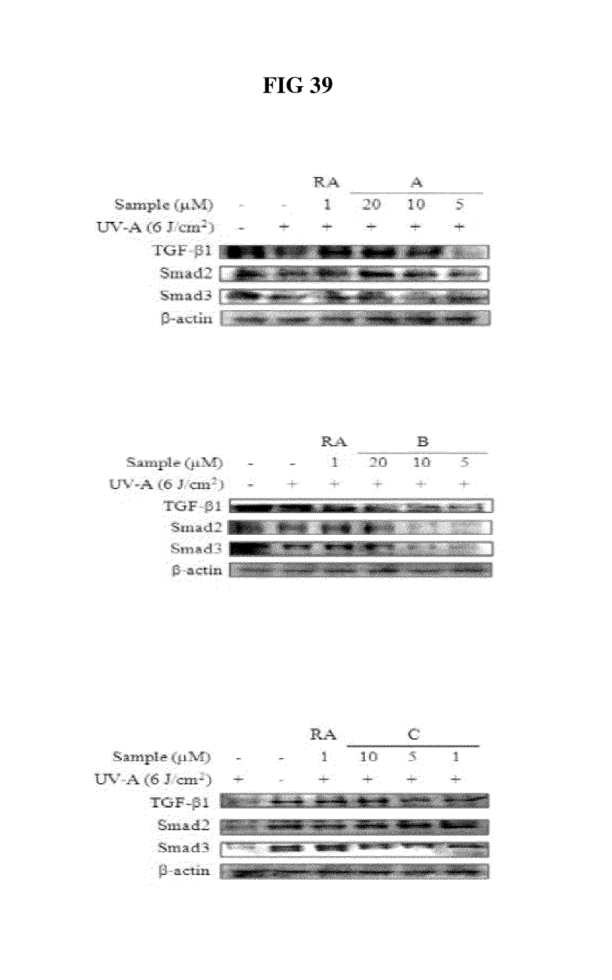

As shown in FIG. 39, TGF-β expression was significantly increased in presence of chromenes in a concentration-dependent manner and their effects were similar with retinoic acid treatment. Unlike TGF-β, AP-1 inhibits positive regulation of procollagen gene transcription of Smad 2, 3 and 4. Therefore, effects of chromenes on Smads protein expression were observed in UV-A exposed dermal fibroblasts compared with retinoic acid treatment group. The result obtained from Western blot analysis showed that the levels of Smad 2 and Smad 3 proteins were significantly increased by treatment with chromenes in UV-A irradiated dermal cells. According to these results, UV-A irradiation causes an impairment of the TGF-β/Smad signaling cascade. This inhibition may also contribute to UV-A mediated reduction of type I collagen synthesis. The mechanisms of protecting of dermal fibroblast by UV-A induced photoaging process is suppressed via TGF-β/Smad signaling cascade by chromenes treatment.

### Experimental Example 20

### Chromenes Regulated AP-1 and TGF-β/Samd Signaling Pathways in UV-A Dependent

Above results demonstrated that chromenes reduced UV-A induced fibroblast damage through the regulation of aging-related gene expression. However, these effects of chromenes were not enough to support above results strongly. Thus, we also checked the AP-1 and TGF-β/smad signaling pathways whether they are regulated by chromenes in non UV-A irradiated fibroblasts.

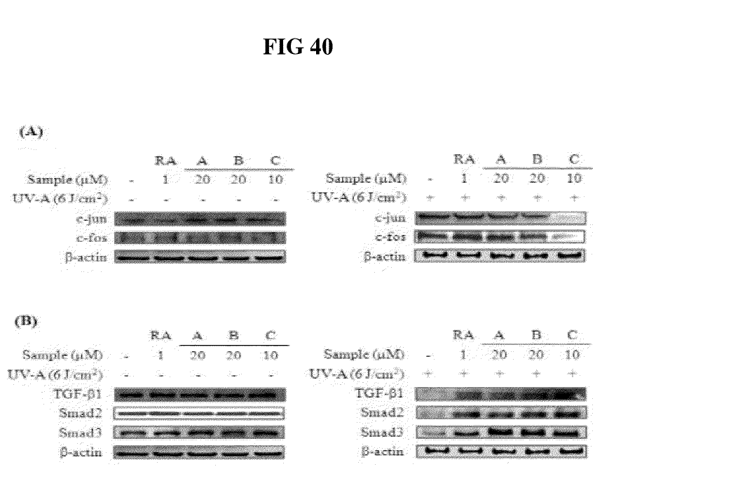

Effects of chromenes on both UV-A irradiated and unirradiated conditions were investigated to improve the results obtained from UV dependant effect of chromene compounds. As shown in FIG. 40, the levels of c-jun, c-fos, TGF-β, Smad 2 and Samd 3 were not affected significantly in the presence of chromenes and retinoic acid without any UV-A exposure. There is no observable difference on their expression levels with compound treatment compared to non-treated groups. As a part of defining the UV-A dependency fibroblasts were investigated with UV-A exposure in addition. Stimulation of dermal fibroblasts with UV-A resulted in suppression of TGF-β/Smad pathway factors whereas AP-1 signaling was increased. However, treatment with chromenes enhanced TGF-β/Smad signaling and reduced AP-1 translation.

Among the compounds, especially compound C regulated the TGF-β/smad and AP-1 related factors in a more efficient way than that of retinoic acid. Taken together, chromenes induced TGF-β/Smad activation followed by reduction of AP-1 activities. Considering the pathway protein regulations of chromenes under UV-A exposure and the absence of any change in TGF-β/Smad and AP-1 pathway factors without UV-A irradiation whilst chromene compounds treatment clearly points out an UV-A dependent activity. In short, these observed effects indicate that effects of compounds on TGF-β/Smad and AP-1 pathways responsible for the protection of cells due to UV-A irradiation.

As set forth above, according to exemplary embodiments of the invention, chromenes derived from Sargassum horneri according to the present invention stimulate a synthesis of collagen and suppress a decomposition of collagen, and also improve skin wrinkles caused by UV-A by exhibiting activity of inhibiting collagenase, elastase, and matrix metalloproteinases. Accordingly, the composition according to the present invention can be used for preventing aging caused by UV light, and specifically, can be used as a composition, foods, food additives, and cosmetic additives for preventing or treating photoaging. Particularly, since Sargassum horneri is a natural material and also edible, it has an advantage in that the composition of the present invention including the compounds derived from the same as an effective component is stable even if it is used for a long period time.

While the present invention has been shown and described in connection with the exemplary embodiments, it will be apparent to those skilled in the art that modifications and variations can be made without departing from the spirit and scope of the invention as defined by the appended claims.

What is claimed is:

1. A composition for inhibiting photoaging of human skin comprising chromenens selected from the group consisting of sargachromenol E, sargachromenol D, and a mixture thereof derived from Sargassum horneri as an effective component.

2. The composition according to claim 1, improving skin wrinkles caused by UV-A.

3. The composition according to claim 1, stimulating a synthesis of collagen and inhibiting a decomposition of collagen.

4. The composition according to claim 1, having activities of inhibiting collagenase, elastase, and matrix metalloproteinases in dermal fibroblasts.

5. The composition according to claim 1, inhibiting transcriptions of AP-1 and TGF-β/smad signaling cascade.

6. The composition according to claim 1, wherein the chromenes are obtained from organic solvent fractions of methanol crude extracts of Sargassum horneri.

7. The composition according to claim 1, wherein the chromenes are sargachromenol D.

8. A method for protecting the skin photoaging in the skin of a mammal exposed to ultraviolet comprising administering to the mammal in need thereof chromenens selected from the group consisting of sargachromenol E, sargachromenol D, and a mixture thereof derived from Sargassum horneri as an effective component.
